(19) United States
(12) Patent Application Publication
(10) Pub. No.: US 2021/0321868 A1
(43) Pub. Date: Oct. 21, 2021

(54) DEVICES AND METHODS FOR DETERMINING A LENS POSITION FOR COMPENSATION OF HIGHER-ORDER ABERRATIONS

(71) Applicant: Ovitz Corporation, Rochester, NY (US)

(72) Inventors: Nicolas Scott BROWN, Rochester, NY (US); Richard E MUNSON, Macedon, NY (US)

(73) Assignee: Ovitz Corporation, Rochester, NY (US)

(21) Appl. No.: 17/233,052

(22) Filed: Apr. 16, 2021

Related U.S. Application Data
(60) Provisional application No. 63/011,981, filed on Apr. 17, 2020.

(51) Int. Cl. A61B 3/00; A61B 3/10; A61B 3/14

(52) U.S. Cl. CPC A61B 3/0025; A61B 3/0058; A61B 3/14; A61B 3/1015; A61B 3/0033

(57) ABSTRACT

A method includes providing an image of at least a portion of an eye wearing a lens with a plurality of reference markings to a display device for display by the display device. The image includes at least a pupil of the eye and at least a subset of the plurality of reference markings. The method further includes receiving one or more of: a first user input regarding two or more periphery reference markings, a second user input regarding one or more angular reference markings, or a third user input for identifying a position reference point of the eye. The method also includes obtaining a lens surface profile that is determined based at least in part on the one or more of the first user input, the second user input, and the third user input.

### RELATED APPLICATIONS

This application claims the benefit of, and priority to, U.S. Provisional Patent Application Ser. No. 63/011,981, filed Apr. 17, 2020, which is incorporated by reference herein in its entirety.

This application is related to U.S. patent application Ser. No. 16/558,298, filed Sep. 2, 2019, which claims the benefit of and priority to U.S. Provisional Patent Application Ser. No. 62/725,305, filed Aug. 31, 2018, both of which are incorporated by reference herein in their entireties.

### TECHNICAL FIELD

This relates generally to determining a position of a corrective lens on an eye, and more specifically to devices and methods for determining a position of a corrective lens used for compensation of higher-order aberrations.

### BACKGROUND

Eyes are important organs, which play a critical role in human's visual perception. An eye has a roughly spherical shape and includes multiple elements, such as cornea, lens, vitreous humour, and retina. Imperfections in these components can cause reduction or loss of vision. For example, too much or too little optical power in the eye can lead to blurring of the vision (e.g., near-sightedness or far-sightedness), and astigmatism can also cause blurring of the vision.

Corrective lenses (e.g., glasses and contact lenses) are frequently used to compensate for blurring caused by too much or too little optical power and/or astigmatism. However, when eyes have higher-order aberrations (e.g., aberrations higher than astigmatism in the Zernike polynomial model of aberrations), conventional corrective lenses have not been effective at compensating for all of the aberrations associated with the eyes, resulting in blurry images even when corrective lenses are used.

### SUMMARY

Accordingly, there is a need for corrective lenses that can compensate for higher-order aberrations. However, there is a variation in the structure and orientation of an eye between patients (and even different eyes of a same patient), and thus, a contact lens placed on an eye will settle in different positions and orientations for different patients (or different eyes). Proper alignment of the corrective lens to the patient's eye is required in order to provide an accurate correction or compensation of the higher-order aberrations in the eye. Thus, the position (e.g., lateral displacements and orientation) information for a contact lens is required along with vision information for effective correction or compensation of the higher-order aberrations in the eye, and devices and methods that can provide the position information along with the vision information are needed.

The above deficiencies and other problems associated with conventional devices and methods are reduced or eliminated by the devices and methods described herein.

In accordance with some embodiments, a method is performed at an electronic device with one or more processors and memory. The electronic device is in communication with a display device. The method includes providing, to the display device for display by the display device, an image of at least a portion of the eye while the user is wearing a lens with a plurality of reference markings on the eye. The image includes at least a pupil of the eye and at least a subset of the plurality of reference markings. The method also includes receiving one or more of a first user input regarding two or more periphery reference markings, a second user input regarding one or more angular reference markings, or a third user input for identifying a position reference point of the eye. The method also includes obtaining a lens surface profile that is determined based at least in part on the one or more of the first user input, the second user input, and the third user input.

In accordance with some embodiments, an electronic device in communication with a display device includes one or more processors and memory storing one or more programs. The one or more programs include instructions for providing, to the display device for display by the display device, a first image of at least a portion of the eye wearing a lens with a plurality of reference markings. The first image includes at least a pupil of the eye and at least a subset of the plurality of reference markings. The one or more programs also include instructions for receiving one or more of: a first user input regarding two or more periphery reference markings; a second user input regarding one or more angular reference markings; or a third user input for identifying a position reference point of the eye. The one or more programs further include instructions for obtaining a lens surface profile determined based at least in part on the one or more of the first user input, the second user input, and the third user input.

In accordance with some embodiments, a computer readable storage medium stores one or more programs. The one or more programs include instructions, which, when executed by one or more processors of an electronic device in communication with a display device, cause the electronic device to provide, to the display device for display by the display device, a first image of at least a portion of the eye wearing a lens with a plurality of reference markings. The first image includes at least a pupil of the eye and at least a subset of the plurality of reference markings. The one or more programs also include instructions, which, when executed by the one or more processors, cause the electronic device to receive one or more of: a first user input regarding two or more periphery reference markings; a second user input regarding one or more angular reference markings; or a third user input for identifying a position reference point of the eye. The one or more programs further include instructions, which, when executed by the one or more processors, cause the electronic device to obtain a lens surface profile determined based at least in part on the one or more of the first user input, the second user input, and the third user input.

In accordance with some embodiments, an electronic device is in communication with a display device. The electronic device includes one or more processors, and memory storing one or more programs. The one or more programs include instructions for performing any method described herein.

In accordance with some embodiments, a computer readable storage medium stores one or more programs. The one or more programs include instructions, which, when executed by one or more processors of an electronic device in communication with a display device, cause the electronic device to perform any method described herein.

Thus, the disclosed embodiments provide methods of collecting position information, which can be used to determine a position of a position reference point (e.g., a visual axis) of an eye relative to a contact lens (or vice versa), in conjunction with vision information. Such information, in turn, allows design and manufacturing of customized (e.g., personalized) contact lenses that can compensate for higher-order aberrations in a particular eye.

### BRIEF DESCRIPTION OF THE DRAWINGS

For a better understanding of the various described embodiments, reference should be made to the Description of Embodiments below, in conjunction with the following drawings in which like reference numerals refer to corresponding parts throughout the figures.

FIG. 1A is a schematic diagram showing a system for vision characterization in accordance with some embodiments.

FIGS. 1B and 1C illustrate optical components of an optical device in accordance with some embodiments.

FIG. 1D illustrates wavefront sensing with the optical device shown in FIGS. 1B and 1C, in accordance with some embodiments.

FIG. 1E illustrates imaging with the optical device shown in FIGS. 1B and 1C, in accordance with some embodiments.

FIGS. 1F and 1G illustrate optical components of an optical device in accordance with some other embodiments.

FIG. 1H is a front view of a measurement instrument in accordance with some embodiments.

FIG. 2 is a block diagram illustrating electronic components of an optical device in accordance with some embodiments.

FIGS. 3A-3D are schematic diagrams illustrating correction of higher-order aberrations in accordance with some embodiments.

FIG. 3E shows a workflow logic for interactive use of a system for generating custom lens fabrication file in accordance with some embodiments.

FIG. 4 shows a user interface for initial verification of wavefront imaging in accordance with some embodiments.

FIGS. 5A and 5B show a user interface for identification of lens markings for perimeter identification in accordance with some embodiments.

FIG. 6 shows a user interface for identification of lens markings for angular orientation in accordance with some embodiments.

FIG. 7 shows a user interface for identifying a position reference point of an eye in accordance with some embodiments.

FIG. 8 shows a user interface for practitioner review, reporting, and file generation in accordance with some embodiments.

FIGS. 9A-9C illustrate a flow diagram representing a method for characterizing an eye of a user in accordance with some embodiments.

These figures are not drawn to scale unless indicated otherwise.

### DETAILED DESCRIPTION

Reference will be made to embodiments, examples of which are illustrated in the accompanying drawings. In the following description, numerous specific details are set forth in order to provide a thorough understanding of the various described embodiments. However, it will be apparent to one of ordinary skill in the art that the various described embodiments may be practiced without these particular details. In other instances, methods, procedures, components, circuits, and networks that are well-known to those of ordinary skill in the art are not described in detail so as not to unnecessarily obscure aspects of the embodiments.

It will also be understood that, although the terms first, second, etc. may be used herein to describe various elements, these elements should not be limited by these terms. These terms are only used to distinguish one element from another. For example, a first image sensor could be termed a second image sensor, and, similarly, a second image sensor could be termed a first image sensor, without departing from the scope of the various described embodiments. The first image sensor and the second image sensor are both image sensors, but they are not the same image sensor.

The terminology used in the description of the embodiments herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting (the stated condition or event)” or “in response to detecting (the stated condition or event),” depending on the context.

A corrective lens (e.g., contact lens) designed to compensate for higher-order aberrations of an eye needs accurate positioning on an eye. If a corrective lens designed to compensate for higher-order aberrations of an eye is not placed accurately, the corrective lens may not be effective in compensating for higher-order aberrations of the eye and may even exacerbate the higher-order aberrations.

One of the additional challenges is that when a corrective lens (e.g., contact lens) is used to compensate for higher-order aberrations of an eye, an apex of a corrective lens is not necessarily positioned on a visual axis of the eye. Thus, a relative position between the visual axis of the eye and the apex of the corrective lens needs to be reflected in the design of the corrective lens. This requires accurate measurements of the visual axis of the eye and a position of the corrective lens on the eye. However, because the eye has a curved three-dimensional surface, conventional methods for determining the position of the corrective lens relative to the visual axis of the eye often have errors. Such errors hamper the performance of a corrective lens designed to compensate for higher-order aberrations. Thus, for designing a corrective lens that can compensate for the higher-order aberrations, an accurate measurement of the visual axis (or any other position reference point) of the eye may be necessary in some cases.

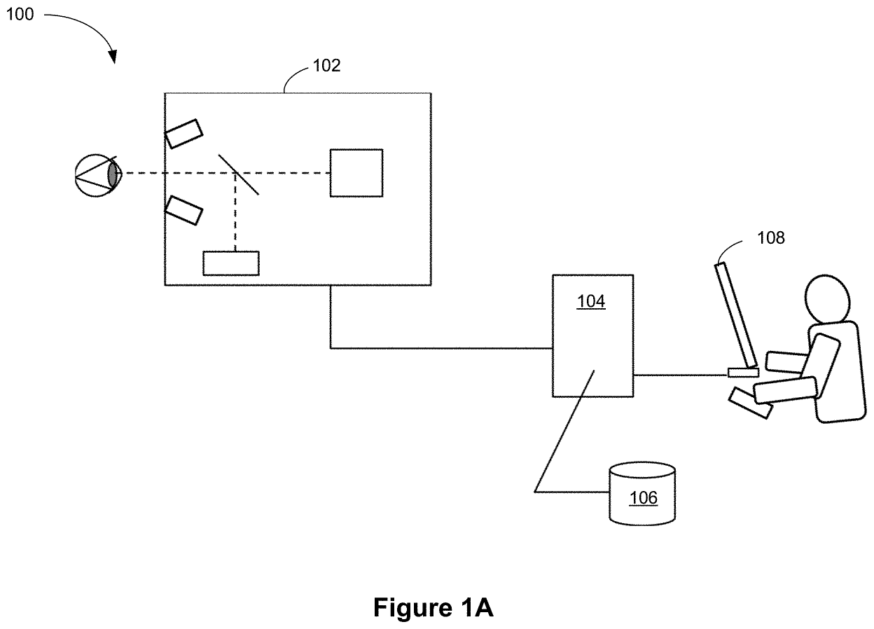

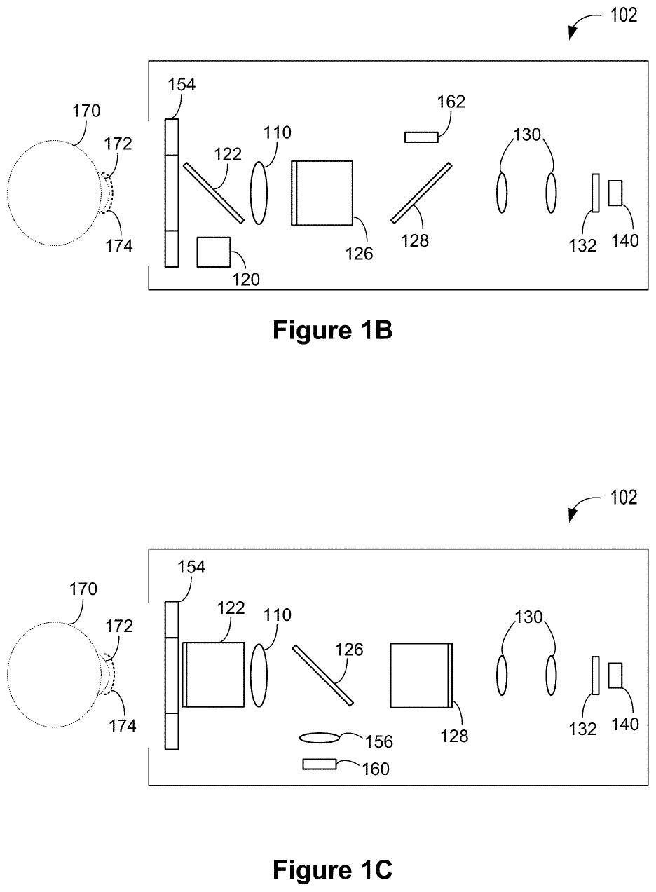

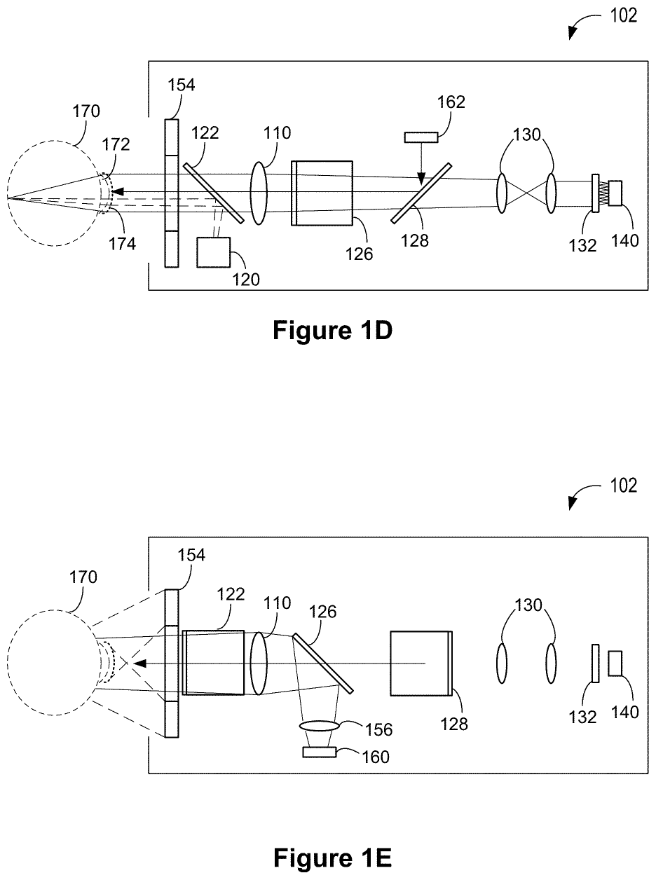

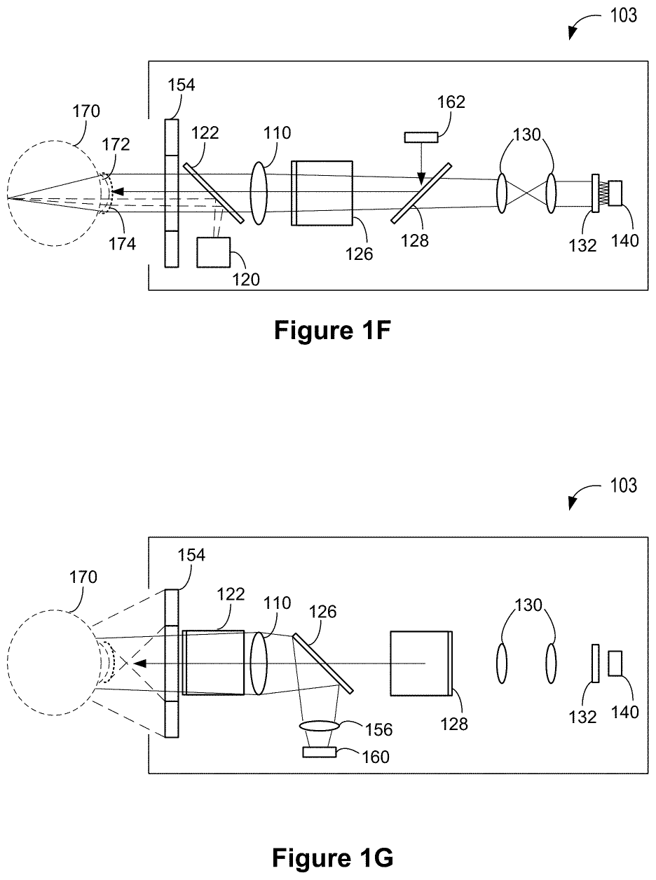

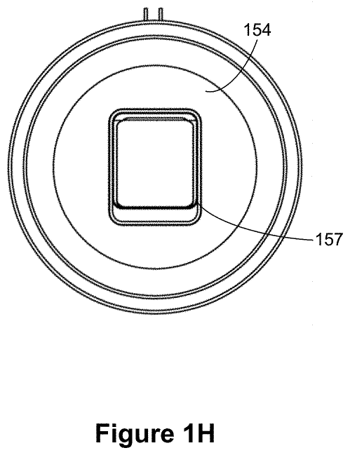

FIG. 1A is a schematic diagram showing a system 100 for vision characterization in accordance with some embodiments. The system 100 includes a measurement device 102, a computer system 104, a database 106, and a display device 108. The measurement device 102 performs a vision characterization of an eye of a patient and provides imaging results and vision profile metrics of the characterized eye. The measurement device 102 includes a wavefront measurement device, such as a Shack-Hartmann wavefront sensor, that is configured to perform wavefront measurements. The display device 108 shows the imaging results and vision profile metrics acquired by the measurement device 102. In some cases, the display device 108 may provide a user (e.g., operator, optometrist, viewer, or practitioner) with one or more options or prompts to correct, validate, or confirm displayed results. The database 106 stores imaging results and vision profile metrics acquired by the measurement device 102 as well as any verified information provided by the user of the system 100. In response to receiving the results from the measurement device 102 and validation of displayed results from the user, the system 100 may generate a correction lens (e.g., contact lens) fabrication file for the patient that is stored in the database 106.

The computer system 104 may include one or more computers or central processing units (CPUs). The computer system 104 is in communication with each of the measurement device 102, the database 106, and the display device 108.

FIGS. 1B-1E illustrate optical components of the measurement device 102 in accordance with some embodiments. FIG. 1B shows a side view (e.g., a side elevational view) of the optical components of the measurement device 102, and FIG. 1C is a top view (e.g., a plan view) of the optical components of the measurement device 102. One or more lenses 156 and second image sensor 160 shown in FIG. 1C are not shown in FIG. 1B to avoid obscuring other components of the measurement device 102 shown in FIG. 1B. In FIG. 1C, pattern 162 is not shown to avoid obscuring other components of the measurement device 102 shown in FIG. 1C.

The measurement device 102 includes lens assembly 110. In some embodiments, lens assembly 110 includes one or more lenses. In some embodiments, lens assembly 110 is a doublet lens. For example, a doublet lens is selected to reduce spherical aberration and other aberrations (e.g., coma and/or chromatic aberration). In some embodiments, lens assembly 110 is a triplet lens. In some embodiments, lens assembly 110 is a singlet lens. In some embodiments, lens assembly 110 includes two or more separate lenses. In some embodiments, lens assembly 110 includes an aspheric lens. In some embodiments, a working distance of lens assembly 110 is between 10-100 mm (e.g., between 10-90 mm, 10-80 mm, 10-70 mm, 10-60 mm, 10-50 mm, 15-90 mm, 15-80 mm, 15-70 mm, 15-60 mm, 15-50 mm, 20-90 mm, 20-80 mm, 20-70 mm, 20-60 mm, 20-50 mm, 25-90 mm, 25-80 mm, 25-70 mm, 25-60 mm, or 25-50 mm). In some embodiments, when the lens assembly includes two or more lenses, an effective focal length of a first lens (e.g., the lens positioned closest to the pupil plane) is between 10-150 mm (e.g., between 10-140 mm, 10-130 mm, 10-120 mm, 10-110 mm, 10-100 mm, 10-90 mm, 10-80 mm, 10-70 mm, 10-60 mm, 10-50 mm, 15-150 mm, 15-130 mm, 15-120 mm, 15-110 mm, 15-100 mm, 15-90 mm, 15-80 mm, 15-70 mm, 15-60 mm, 15-50 mm, 20-150 mm, 20-130 mm, 20-120 mm, 20-110 mm, 20-100 mm, 20-90 mm, 20-80 mm, 20-70 mm, 20-60 mm, 20-50 mm, 25-150 mm, 25-130 mm, 25-120 mm, 25-110 mm, 25-100 mm, 25-90 mm, 25-80 mm, 25-70 mm, 25-60 mm, 25-50 mm, 30-150 mm, 30-130 mm, 30-120 mm, 30-110 mm, 30-100 mm, 30-90 mm, 30-80 mm, 30-70 mm, 30-60 mm, 30-50 mm, 35-150 mm, 35-130 mm, 35-120 mm, 35-110 mm, 35-100 mm, 35-90 mm, 35-80 mm, 35-70 mm, 35-60 mm, 35-50 mm, 40-150 mm, 40-130 mm, 40-120 mm, 40-110 mm, 40-100 mm, 40-90 mm, 40-80 mm, 40-70 mm, 40-60 mm, 40-50 mm, 45-150 mm, 45-130 mm, 45-120 mm, 45-110 mm, 45-100 mm, 45-90 mm, 45-80 mm, 45-70 mm, 45-60 mm, 45-50 mm, 50-150 mm, 50-130 mm, 50-120 mm, 50-110 mm, 50-100 mm, 50-90 mm, 50-80 mm, 50-70 mm, or 50-60 mm). In some embodiments, for an 8 mm pupil diameter, the lens diameter is 16-24 mm. In some embodiments, for a 7 mm pupil diameter, the lens diameter is 12-20 mm. In some embodiments, the f-number of lens assembly is between 2 and 5. The use of a common lens assembly (e.g., lens assembly 110) in both a wavefront sensor and a contact lens center sensor allows the integration of the wavefront sensor and the contact lens center sensor without needing large diameter optics.

The measurement device 102 also includes a wavefront sensor. In some embodiments, the wavefront sensor includes first light source 120, lens assembly 110, an array of lenses 132 (also called herein lenslets), and first image sensor 140. In some embodiments, the wavefront sensor includes additional components (e.g., one or more lenses 130). In some embodiments, the wavefront sensor does not include such additional components.

First light source 120 is configured to emit first light and transfer the first light emitted from the first light source toward eye 170, as depicted in FIG. 1D.

FIGS. 1B-1E include eye 170, its components (e.g., cornea 172), and contact lens 174 to illustrate the operations of the measurement device 102 with eye 170 and contact lens 174. However, eye 170, its components, and contact lens 174 are not part of the measurement device 102.

Turning back to FIG. 1B, in some embodiments, first light source 120 is configured to emit light of a single wavelength or a narrow band of wavelengths. Exemplary first light source 120 includes a laser (e.g., a laser diode) or a light-emitting diode (LED).

In some embodiments, first light source 120 includes one or more lenses to change the divergence of the light emitted from first light source 120 so that the light, after passing through the one or more lenses, is collimated.

In some embodiments, first light source 120 includes a pinhole (e.g., having a diameter of 1 mm or less, such as 400 μm, 500 μm, 600 μm, 700 μm, 800 μm, 900 μm, and 1 mm).

In some cases, an anti-reflection coating is applied on a back surface (and optionally, a front surface) of lens assembly 110 to reduce reflection. In some embodiments, first light source 120 is configured to transfer the first light emitted from first light source 120 off an optical axis of the measurement device 102 (e.g., an optical axis of lens assembly 110), as shown in FIG. 1D (e.g., the first light emitted from first light source 120 propagates parallel to, and offset from, the optical axis of lens assembly 110). This reduces back reflection of the first light emitted from first light source 120, by cornea 172, toward first image sensor 140. In some embodiments, the wavefront sensor includes a quarter-wave plate to reduce back reflection, of the first light, from lens assembly 110 (e.g., light reflected from lens assembly 110 is attenuated by the quarter-wave plate). In some embodiments, the quarter-wave plate is located between beam steerer 122 and first image sensor 140.

First image sensor 140 is configured to receive light, from eye 170, transmitted through lens assembly 110 and the array of lenses 132. In some embodiments, the light from eye 170 includes light scattered at a retina or fovea of eye 170 (in response to the first light from first light source 120). For example, as shown in FIG. 1D, light from eye 170 passes multiple optical elements, such as beam steerer 122, lens assembly 110, beam steerer 126, beam steerer 128, and lenses 130, and reaches first image sensor 140.

Beam steerer 122 is configured to reflect light from light source 120 and transmit light from eye 170, as shown in FIG. 1D. Alternatively, beam steerer 122 is configured to transmit light from light source 120 and reflect light from eye 170. In some embodiments, beam steerer 122 is a beam splitter (e.g., 50:50 beam splitter, polarizing beam splitter, etc.). In some embodiments, beam steerer 122 is a wedge prism, and when first light source 120 is configured to have a linear polarization, the polarization of the light emitted from first light source 120 is configured to reflect at least partly by the wedge prism. Light of a polarization that is orthogonal to the linear polarization of the light emitted from first light source 120 is transmitted through the wedge prism. In some cases, the wedge prism also reduces light reflected from cornea 172 of eye 170.

In some embodiments, beam steerer 122 is tilted at such an angle (e.g., an angle between the optical axis of the measurement device 102 and a surface normal of beam steerer 122 is at an angle less than 45°, such as 30°) so that the space occupied by beam steerer 122 is reduced.

In some embodiments, the measurement device 102 includes one or more lenses 130 to modify a working distance of the measurement device 102.

The array of lenses 132 is arranged to focus incoming light onto multiple spots, which are imaged by first image sensor 140. As in Shack-Hartmann wavefront sensor, an aberration in a wavefront causes displacements (or disappearances) of the spots on first image sensor 140. In some embodiments, a Hartmann array is used instead of the array of lenses 132. A Hartmann array is a plate with an array of apertures (e.g., through-holes) defined therein.

In some embodiments, one or more lenses 130 and the array of lenses 132 are arranged such that the wavefront sensor is configured to measure a reduced range of optical power. A wavefront sensor that is capable of measuring a wide range of optical power may have less accuracy than a wavefront sensor that is capable of measuring a narrow range of optical power. Thus, when a high accuracy in wavefront sensor measurements is desired, the wavefront sensor can be designed to cover a narrow range of optical power. For example, a wavefront sensor for diagnosing low and medium myopia can be configured with a narrow range of optical power between 0 and −6.0 diopters, with its range centering around −3.0 diopters. Although such a wavefront sensor may not provide accurate measurements for diagnosing hyperopia (or determining a prescription for hyperopia), the wavefront sensor would provide more accurate measurements for diagnosing myopia (or determining a prescription for myopia) than a wavefront sensor that can cover both hyperopia and myopia (e.g., from −6.0 to +6.0 diopters). In addition, there are certain populations in which it is preferable to maintain a center of the range at a non-zero value. For example, in some Asian populations, the optical power may range from +6.0 to −14.0 diopters (with the center of the range at−4.0 diopters), whereas in some Caucasian populations, the optical power may range from +8.0 to −12.0 diopters (with the center of the range at−2.0 diopters). The center of the range can be shifted by moving the lenses (e.g., one or more lenses 130 and/or the array of lenses 132). For example, defocusing light from eye 170 can shift the center of the range.

The measurement device 102 further includes a contact lens center sensor (or a corneal vertex sensor). In some embodiments, the contact lens center sensor includes lens assembly 110, second light source 154, and second image sensor 160. In some embodiments, as shown in FIG. 1C, second image sensor 160 is distinct from first image sensor 140. In some embodiments, the wavefront sensor includes additional components that are not included in the contact lens center sensor (e.g., array of lenses 132).

Second light source 154 is configured to emit second light and transfer the second light emitted from second light source 154 toward eye 170. As shown in FIG. 1E, in some embodiments, second light source 154 is configured to transfer the second light emitted from second light source 154 toward eye 170 without transmitting the second light emitted from second light source 154 through lens assembly 110 (e.g., second light from second light source 154 is directly transferred to eye 170 without passing through lens assembly 110).

In some embodiments, the measurement device 102 includes beam steerer 126 configured to transfer light from eye 170, transmitted through lens assembly 110, toward first image sensor 140 and/or second image sensor 160. For example, when the measurement device 102 is configured for wavefront sensing (e.g., when light from first light source 120 is transferred toward eye 170), beam steerer 126 transmits light from eye 170 toward first image sensor 140, and when the measurement device 102 is configured for contact lens center determination (e.g., when light from second light source 154 is transferred toward eye 170), beam steerer 126 transmits light from eye 170 toward second image sensor 160.

Second light source 154 is distinct from first light source 120. In some embodiments, first light source 120 and second light source 154 emit light of different wavelengths (e.g., first light source 120 emits light of 900 nm wavelength, and second light source 154 emits light of 800 nm wavelength; alternatively, first light source 120 emits light of 850 nm wavelength, and second light source 154 emits light of 950 nm wavelength).

In some embodiments, beam steerer 126 is a dichroic mirror (e.g., a mirror that is configured to transmit the first light from first light source 120 and reflect the second light from second light source 154, or alternatively, reflect the first light from first light source 120 and transmit the second light from second light source 154). In some embodiments, beam steerer 126 is a movable mirror (e.g., a mirror that can flip or rotate to steer light toward first image sensor 140 and second image sensor 160). In some embodiments, beam steerer 126 is a beam splitter. In some embodiments, beam steerer 126 is configured to transmit light of a first polarization and reflect light of a second polarization that is distinct from (e.g., orthogonal to) the first polarization. In some embodiments, beam steerer 126 is configured to reflect light of the first polarization and transmit light of the second polarization.

In some embodiments, second light source 154 is configured to project a predefined pattern of light on the eye. In some embodiments, second light source 154 is configured to project an array of spots on the eye. In some embodiments, the array of spots is arranged in a grid pattern.

In some embodiments, second light source 154 includes one or more light emitters (e.g., light-emitting diodes) and diffuser (e.g., a diffuser plate having an array of spots).

FIGS. 1F and 1G illustrate optical components of a measurement instrument 103 in accordance with some other embodiments. Measurement instrument 103 is similar to the measurement device 102 shown in FIGS. 1B-1E except that measurement instrument 103 includes only one lens 130.

FIG. 1H is a front view of the measurement device 102 in accordance with some embodiments. The side view of the measurement device 102 shown in FIG. 1H corresponds to a view of the measurement device 102 seen from a side that is adjacent to second light source 154. In FIG. 1H, the measurement device 102 includes second light source 154, which has a circular shape with a rectangular hole 157 defined in it. Second light source 154 shown in FIG. 1H projects a pattern of light.

Turning back to FIG. 1E, second image sensor 160 is configured to receive light, from eye 170. In some embodiments, the light from eye 170 includes light reflected from cornea 172 of eye 170 (in response to the second light from second light source 154). For example, as shown in FIG. 1E, light from eye 170 (e.g., light reflected from cornea 172) interacts with multiple optical elements, such as lens assembly 110, beam steerer 122, lens 124, beam steerer 126, and one or more lenses 156, and reaches second image sensor 160.

The lenses in the contact lens center sensor (e.g., lens assembly 110 and one or more lenses 156) are configured to image a pattern of light projected on cornea 172 onto second image sensor 160. For example, when a predefined pattern of light is projected on cornea 172, the image of the predefined pattern of light detected by second image sensor 160 is used to determine a center of a contact lens on the cornea (or a vertex of the cornea).

In some embodiments, the measurement device 102 includes pattern 162 and beam steerer 128. Pattern 162 is an image that is projected toward eye 170 to facilitate positioning of eye 170. In some embodiments, pattern 162 includes an image of an object (e.g., balloon), an abstract shape (e.g., a cross), or a pattern of light (e.g., a shape having a blurry edge).

In some embodiments, beam steerer 128 is a dichroic mirror (e.g., a mirror that is configured to transmit the light from eye 170 and reflect light from pattern 162, or alternatively, reflect light from eye 170 and transmit light from pattern 162). In some embodiments, beam steerer 128 is a movable mirror. In some embodiments, beam steerer 128 is a beam splitter. In some embodiments, beam steerer 128 is configured to transmit light of a first polarization and reflect light of a second polarization that is distinct from (e.g., orthogonal to) the first polarization. In some embodiments, beam steerer 128 is configured to reflect light of the first polarization and transmit light of the second polarization.

FIG. 1D illustrates operation of the measurement device 102 for wavefront sensing without operations for determining a contact lens center and FIG. 1E illustrates operation of the measurement device 102 for determining a contact lens center without wavefront sensing. In some embodiments, the measurement device 102 sequentially operates between wavefront sensing and determining a contact lens center. For example, in some cases, the measurement device 102 performs wavefront sensing and subsequently, determines a contact lens center. In some other cases, the measurement device 102 determines a contact lens center, and subsequently performs wavefront sensing. In some embodiments, the measurement device 102 switches between wavefront sensing and determining a contact lens center. In some embodiments, the measurement device 102 repeats wavefront sensing and determining a contact lens center. In some embodiments, the measurement device 102 operates for wavefront sensing concurrently with determining a contact lens center (e.g., light from first light source 120 and light from second light source 154 are delivered toward eye 170 at the same time, and first image sensor 140 and second image sensor 160 collect images at the same time). For brevity, such details are not repeated herein.

In some embodiments, light from pattern 162 is projected toward eye 170 while the measurement device 102 operates for wavefront sensing (as shown in FIG. 1D). In some embodiments, light from pattern 162 is projected toward eye 170 while device operates for determining a contact lens center (as shown in FIG. 1E).

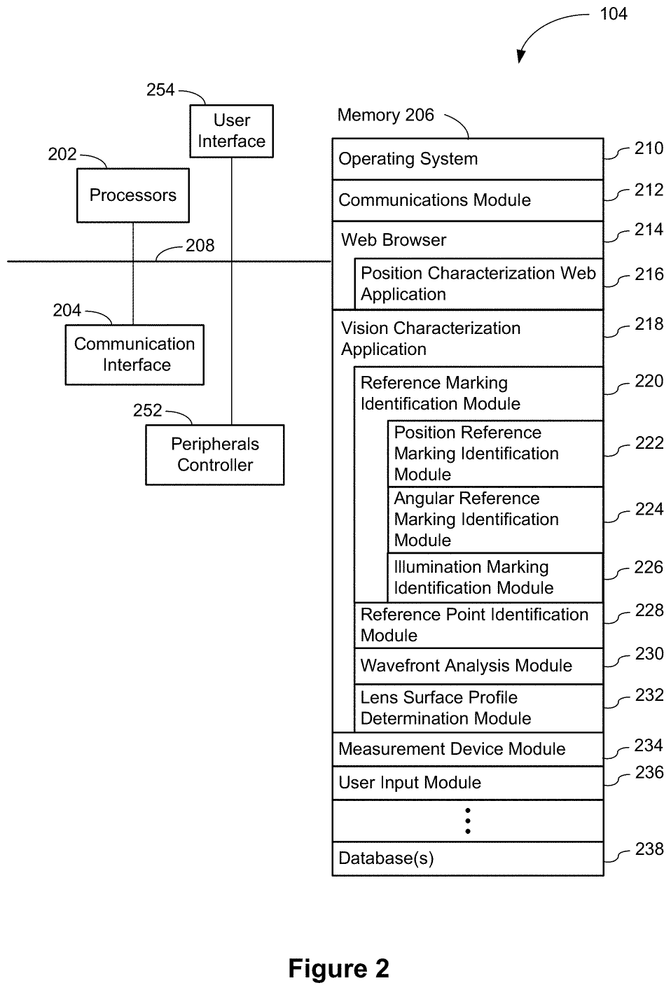

FIG. 2 shows block diagram illustrating electronic components of computer system 104 in accordance with some embodiments. Computer system 104 includes one or more processing units 202 (central processing units, application processing units, application-specific integrated circuit, etc., which are also called herein processors), one or more network or other communications interfaces 204, memory 206, and one or more communication buses 208 for interconnecting these components. In some embodiments, communication buses 208 include circuitry (sometimes called a chipset) that interconnects and controls communications between system components. In some embodiments, system 100 includes a user interface 254 (e.g., a user interface having the display device 108, which can be used for displaying acquired images, one or more buttons, and/or other input devices). In some embodiments, computer system 104 also includes peripherals controller 252, which is configured to control operations of components of the measurement device 102, such as first light source 120, first image sensor 140, second light source 150, and second image sensor 160 (e.g., initiating respective light sources to emit light, and/or receiving information, such as images, from respective image sensors).

In some embodiments, communications interfaces 204 include wired communications interfaces and/or wireless communications interfaces (e.g., Wi-Fi, Bluetooth, etc.).

Memory 206 of computer system 104 includes high-speed random access memory, such as DRAM, SRAM, DDR RAM or other random access solid state memory devices; and may include non-volatile memory, such as one or more magnetic disk storage devices, optical disk storage devices, flash memory devices, or other non-volatile solid state storage devices. Memory 206 may optionally include one or more storage devices remotely located from the processors 202. Memory 206, or alternately the non-volatile memory device(s) within memory 206, comprises a computer readable storage medium (which includes a non-transitory computer readable storage medium and/or a transitory computer readable storage medium). In some embodiments, memory 206 includes a removable storage device (e.g., Secure Digital memory card, Universal Serial Bus memory device, etc.). In some embodiments, memory 206 or the computer readable storage medium of memory 206 stores the following programs, modules and data structures, or a subset thereof:
operating system 210 that includes procedures for handling various basic system services and for performing hardware dependent tasks;
network communication module (or instructions) 212 that is used for connecting computer system 104 to other computers (e.g., clients and/or servers) via one or more communications interfaces 204 and one or more communications networks, such as the Internet, other wide area networks, local area networks, metropolitan area networks, and so on;
vision characterization application 218, or position characterization web application 216 that runs in a web browser 214, that characterizes position information from an image of an eye and markings;
measurement device module 234 that controls operations of the light sources and the image sensors in the measurement device 102 (e.g., for receiving images from the measurement device 102);
user input module 236 configured for handling user inputs on computer system 104 (e.g., pressing of buttons on computer system 104 or pressing of buttons on a user interface, such as a keyboard, mouse, or touch-sensitive display, that is in communication with computer system 104); and
one or more databases 238 (e.g., database 106) that store information acquired by the measurement device 102.

In some embodiments, memory 206 also includes one or both of:
user information (e.g., information necessary for authenticating a user of computer system 104); and
patient information (e.g., optical measurement results and/or information that can identify patients whose optical measurement results are stored in the one or more databases 238 on computer system 104).

In some embodiments, vision characterization application 218, or vision characterization web application 216, includes the following programs, modules and data structures, or a subset or superset thereof:
reference marking identification module 220 configured for identifying (e.g., automatically identifying) one or more reference markings in an image captured (e.g., recorded, acquired) by the measurement device 102, which may include one or more of the following:
   periphery reference marking identification module 222 configured for identifying (e.g., automatically identifying) one or more periphery reference markings in an image captured (e.g., recorded, acquired) by the measurement device 102;
   angular reference marking identification module 224 configured for identifying (e.g., automatically identifying) one or more angular reference markings in an image captured (e.g., recorded, acquired) by the measurement device 102; and
   illumination marking identification module 226 configured for identifying (e.g., automatically identifying) one or more illumination markings in an image captured (e.g., recorded, acquired) by the measurement device 102;
reference point identification module 228 configured for identifying (e.g., automatically identifying) a position reference point of a patient's eye based on an image captured (e.g., recorded, acquired) by the measurement device 102;
wavefront analysis module 230 configured for analyzing the wavefront measured for a patient's eye(s) using the measurement device 102; and
lens surface profile determination module 232 configured for determining a lens surface profile for a patient's eye(s) based the wavefront measured for a patient's eye and the positions of reference markings.

In some embodiments, wavefront analysis module 230 includes the following programs and modules, or a subset or superset thereof:
an analysis module configured for analyzing images received from first image sensor 140; and
a first presentation module configured for presenting measurement and analysis results from first analysis module (e.g., graphically displaying images received from first image sensor 140, presenting aberrations shown in images received from first image sensor 140, sending the results to another computer, etc.).

In some embodiments, measurement device module 234 includes the following programs and modules, or a subset or superset thereof:
a light source module configured for initiating first light source 120 (through peripherals controller 252) to emit light;
an image sensing module configured for receiving images from first image sensor 140;
a light source module configured for initiating second light source 154 (through peripherals controller 252) to emit light;
an image sensing module configured for receiving images from second image sensor 160;
an image acquisition module configured for capturing one or more images of a patient's eye(s) using the measurement device 102; and
an image stabilization module configured for reducing blurring during acquisition of images by image sensors.

In some embodiments, the computer system 104 may include other modules such as:
an analysis module configured for analyzing images received from second image sensor 160 (e.g., determining a center of a projected pattern of light);
a presentation module configured for presenting measurement and analysis results from second analysis module (e.g., graphically displaying images received from second image sensor 160, presenting cornea curvatures determined from images received from second image sensor 160, sending the results to another computer, etc.);
a spot array analysis module configured for analyzing spot arrays (e.g., measuring displacements and/or disappearances of spots in the spot arrays); and
a centering module configured for determining a center of a projected pattern of light.

In some embodiments, a first image sensing module initiates execution of the image stabilization module to reduce blurring during acquisition of images by first image sensor 140, and a second image sensing module initiates execution of the image stabilization module to reduce blurring during acquisition of images by second image sensor 160.

In some embodiments, a first analysis module initiates execution of spot array analysis module to analyze spot arrays in images acquired by first image sensor 140, and a second analysis module initiates execution of spot array analysis module to analyze spot arrays in images acquired by second image sensor 160.

In some embodiments, a first analysis module initiates execution of spot array analysis module to analyze spot arrays in images acquired by first image sensor 140, and a second analysis module initiates execution of centering module to analyze images acquired by second image sensor 160.

In some embodiments, the one or more databases 238 may store any of: wavefront image data, including information representing the light received by the first image sensor (e.g., images received by the first image sensor), and pupil image data, including information representing the light received by the second image sensor (e.g., images received by the second image sensor).

Each of the above identified modules and applications correspond to a set of instructions for performing one or more functions described above. These modules (i.e., sets of instructions) need not be implemented as separate software programs, procedures or modules, and thus various subsets of these modules may be combined or otherwise re-arranged in various embodiments. In some embodiments, memory 206 may store a subset of the modules and data structures identified above. Furthermore, memory 206 may store additional modules and data structures not described above.

Notwithstanding the discrete blocks in FIG. 2, these figures are intended to be a functional description of some embodiments, although, in some embodiments, the discrete blocks in FIG. 2 can be a structural description of functional elements in the embodiments. One of ordinary skill in the art will recognize that an actual implementation might have the functional elements grouped or split among various components. In practice, and as recognized by those of ordinary skill in the art, items shown separately could be combined and some items could be separated. For example, in some embodiments, measurement device module 234 is part of vision characterization application 218 (or vision characterization web application 216). In other embodiments, reference marking identification module 220, wavefront analysis module 230, and lens surface profile determination module 232 are implemented as separate applications. In some embodiments, one or more programs, modules, or instructions may be implemented in measurement device 102 instead of computer system 104.

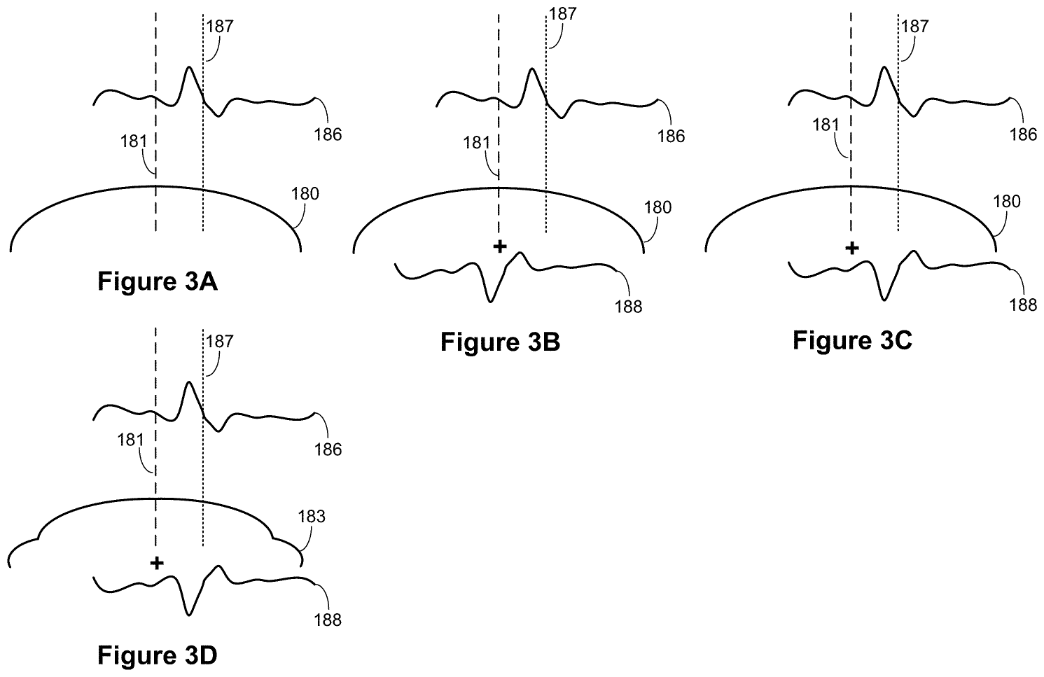

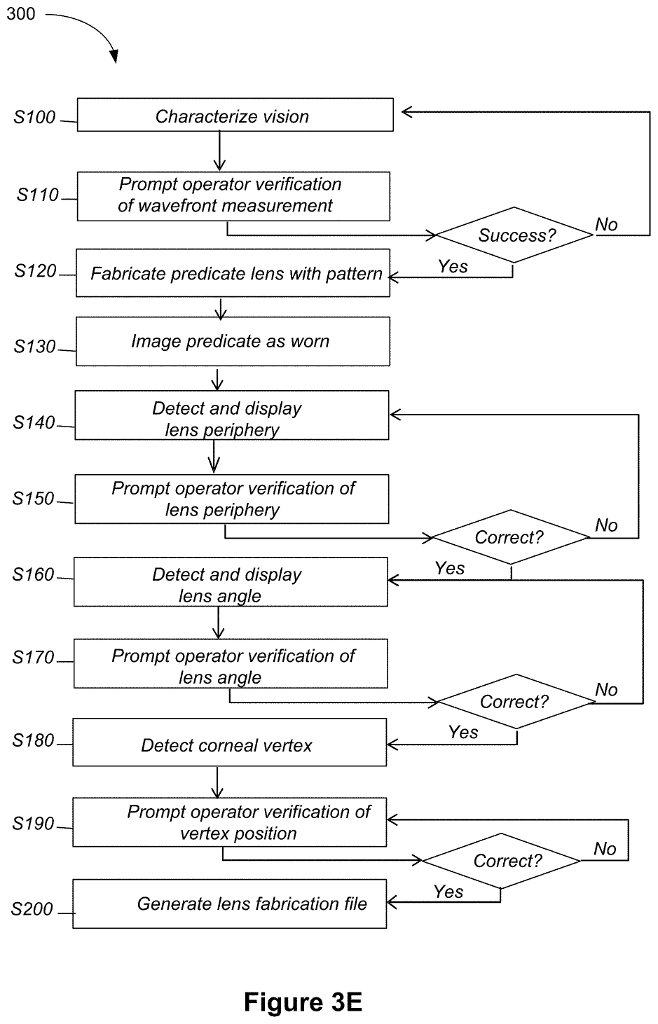

FIGS. 3A-3D are schematic diagrams illustrating correction of higher-order aberrations in accordance with some embodiments.

FIG. 3A illustrates a surface profile of a contact lens 180 without higher-order correction. As a result, an eye wearing the contact lens 180 may see higher-order aberrations represented by line 186. The visual axis 187 of the eye is typically not aligned with the centerline 181 of the contact lens 180, and thus, the measured higher-order aberrations are not aligned with the center of the contact lens 180.

FIG. 3B illustrates modification of the surface profile of the contact lens 180 by superposing a surface profile 188 configured to compensate for the higher-order aberrations. However, when the surface profile 188 is positioned around the centerline 181 of the contact lens 180 as shown in FIG. 3B, the combined surface profile is not effective in reducing the higher-order aberrations, as the surface profile 188 is offset from the higher-order aberrations measured along the visual axis 187 of the eye.

FIG. 3C illustrates modification of the surface profile of the contact lens 180 by superposing the surface profile 188 configured to compensate for the higher-order aberrations where the surface profile 188 is positioned around the visual axis 187 of the eye instead of the centerline 181 of the contact lens 180. By modifying the surface profile of the contact lens 180 by superposing the surface profile 188 with an offset (e.g., the surface profile 188 is in line with the visual axis 187 of the eye), a lens with the modified surface profile can better compensate for higher-order aberrations.

FIG. 3D is similar to FIG. 3C except that the modification of the surface profile can be applied to a multifocal lens 183.

FIG. 3E is a flow diagram for a method 300 for interactive use of a vision characterization system, such as system 100, for generating a customized lens fabrication file in accordance with some embodiments. At step S100, the system 100 performs a characterization of the eye, by using a method, such as a wavefront sensing (e.g., using a Shack-Hartmann wavefront sensor). In some embodiments, step S100 is followed by step S110, which includes prompting an operator of the vision characterization system 100 to verify that the vision characterization (e.g., wavefront assessment) was successful. Once the results of the wavefront characterization results are verified or deemed to be usable, a predicate lens is prepared at step S120. In some cases, the predicate lens is a temporary fixture that is designed for the eye of the individual patient based on the wavefront characterization results. In some cases, the predicate lens is fabricated independent of the wavefront characterization results (e.g., the predicate lens is a reference lens fabricated before the wavefront sensing, may or may not have any optical power, and may or may not have any pattern for correcting higher-order aberrations). The predicate lens has markings that serve as useful references or indices for lens translation and rotation with respect to a visual axis of the patient's eye. In some embodiments, the wavefront characterization is performed using the measurement device 102 and a user (e.g., operator) of the vision characterization system 100 may provide one or more user inputs at an input device that is part of or in communication with vision characterization system 100 (e.g., via a mouse, keyboard, or touch sensitive display) to verify that the wavefront characterization was successful.

The patient wears the predicate lens, and an image of the patient's eye wearing the predicate lens is obtained using the measurement device 102 at step S130.

A position of the predicate lens is determined in step S140 by identifying one or more reference markings (e.g., one or more periphery reference markings) on the predicate lens that correspond to a periphery portion of the predicate lens. Identification of the one or more periphery reference markings, and therefore determining of the position of the predicate lens, may include an automated process, a manual entry process, or a combination of an automated process and a manual verification/correction process. For example, position(s) of the one or more periphery reference markings may be identified via an automated process (e.g., independently of any user input). In some embodiments, the automatically identified position(s) of the one or more periphery reference markings are provided (e.g., displayed, shown) to the user via a display device (e.g., display device 108) that is part of or in communication with system 100.

In step S150, the vision characterization system 100 prompts the user to correct or verify the independently (e.g., automatically) identified position(s) of the one or more periphery reference markings. For example, after position(s) of the one or more periphery reference markings are identified via an automated process (e.g., independently of any user input), the user may be prompted to provide one or more user inputs regarding the automatically identified positions of the one or more periphery reference markings. The one or more user inputs may include a user input that accepts the independently identified positions or each of the one or more periphery reference markings. In some embodiments, such as when there is an error in marking placement, the vision characterization system 100 may fail to identify at least a position of the one or more periphery reference markings. In such cases, the user may provide a user input that instructs the vision characterization system 100 to re-attempt to automatically identify position(s) of the one or more periphery reference markings. The operator may also provide one or more user inputs that corrects incorrectly identified position(s) of one or more periphery reference markings or identifies positions(s) of one or more periphery reference markings that the system 100 failed to automatically identify or detect.

An orientation of the predicate lens is determined in step S160 by identifying one or more angular reference markings on the predicate lens. The one or more angular reference markings are located along the lens periphery. The one or more angular markings are formed in a distinctive pattern in order to provide non-ambiguous identification of an orientation (e.g., a rotation, angle, or angular orientation) of the predicate lens. For example, marking pattern (e.g., one or more dots) may be formed at a 12 o'clock position of the predicate lens to provide a zero-degree reference indicator. Alternately, the marker pattern can be distinctive at two or more locations that combine to provide angular reference, such as providing distinctive markers at 4 and 8 o'clock positions of the predicate lens. Identification of the one or more angular reference markings, and therefore determining of the orientation of the predicate lens, may include an automated process, a manual entry process, or a combination of an automated process and a manual verification/correction process. For example, a position of an angular reference marking may be identified via an automated process (e.g., independently of any user input). In some embodiments, the automatically identified positions of the angular reference markings are provided (e.g., displayed, shown) to the user via a display device (e.g., display device 108) that is part of or in communication with vision characterization system 100.

The vision characterization system 100 may prompt the operator to correct or verify the independently (e.g., automatically) identified position(s) of the one or more angular reference markings in step S170. Details regarding user input corresponding to the automatically identified position(s) of the one or more angular markings are similar to the process described in step S140 with respect to the one or more periphery reference markings and thus, such details are not repeated herein for brevity.

Detection and verification of the corneal vertex are performed in steps S180 and S190, respectively. The vertex of the cornea is a point that is along or very close to the visual axis. The visual axis may not (and typically does not) coincide with the pupil center, but in some cases, can be approximated using the vertex of the cornea and pupil center.

In step S180, a position of the vertex of the cornea is identified via identification of the positions of illumination markings as shown in the image of the eye (e.g., illumination markings corresponding to reflections of illumination sources in the image of the eye). Determination of the positions of the illumination markings, and therefore determination of the position of the vertex of the cornea, may include an automated process, a manual entry process, or a combination of an automated process and a manual verification/correction process. For example, positions of the illumination markings may be identified via an automated process (e.g., independently of any user input). In some embodiments, the automatically identified position(s) of the one or more illumination markings are provided (e.g., displayed) to the user via a display device (e.g., display device 108) that is part of or in communication with vision characterization system 100.

In step S190, the vision characterization system 100 may prompt the user to correct or verify the independently (e.g., automatically) identified position of the illumination markings and/or the vertex of the cornea (which is identified based on the positions of the illumination markings). Details regarding user input corresponding to the automatically identified positions of the illumination markings and/or the vertex of the cornea are similar to the process described in step S140 with respect to the one or more periphery reference markings and thus, the details are not repeated here for brevity.

Once the position(s) of the one or more illumination markings, the positions of the angular reference markings, and/or the position of the vertex of the cornea are determined (e.g., determined and verified), a lens fabrication file that defines a corrective lens designed specifically for the patient's imaged eye is generated in step S200.

In practice, the order of the steps may be different from that described here. For example, although operations regarding determination (e.g., identification and verification) of the position(s) of the one or more periphery reference markings (steps S140 and S150) are shown as occurring before determination of the positions of the angular reference markings (steps S160 and S170) and determination of the position of the vertex of the cornea (steps S180 and S190), each identification step and verification step can occur in any order. Any of steps S140, S160, and S180 may occur prior to any of steps S150, S170, and S190. For example, the position of the vertex of the cornea may be identified (step S180) prior to or simultaneously with identification of the positions of the angular reference markings (step S160). In another example, steps S140, S160, and S180 may occur prior to step S170.

In some embodiments, the patient may be positioned at the measurement device 102 (as shown in FIGS. 1A-1G) during a portion of the operations in the method 300 or during the entire operations in the method 300. For example, the patient may be positioned at the measurement device 102 during the operation of steps S100 and S130 and other steps in the method 300 may be executed after sufficient images of the patient's eye(s) have been acquired (e.g., a few minutes after, hours after, days after, weeks after). In some configurations, one or more of steps S140, S160, S180, and S200 may be performed at a device that is located remotely from the measurement device 102 (e.g., the computer system 104 located remotely from the measurement device 102 receives an image of an eye wearing a lens with markings and performs steps S140, S160, S180, and S200). In another example, the patient may be positioned at the measurement device 102 during the entire operation.

In some embodiments, the method 300 may not include any prompting operation (e.g., S110, S150, S170, and S190). In such cases, the vision characterization system 100 determines positions of the markings automatically (e.g., independent of user input or confirmation).

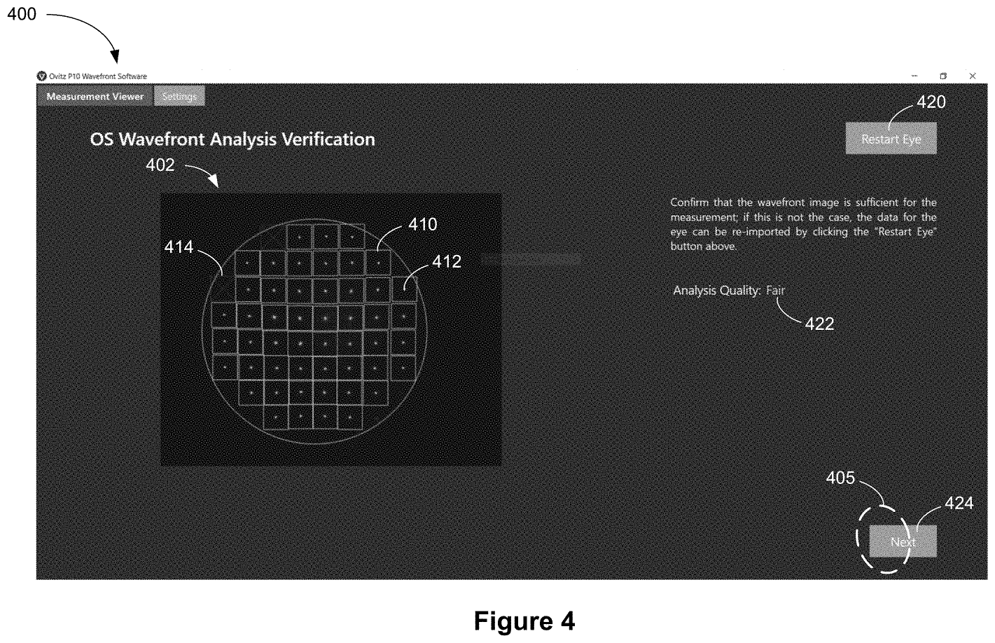

FIGS. 4-8 illustrate user interfaces displayed on display device 108 while certain operations of the method 300 described with respect to FIG. 3E are performed in accordance with some embodiments.

FIG. 4 illustrates user interface 400 displayed on display device 108 in accordance with some embodiments. User interface 400 may be provided to an operator of a vision characterization system (e.g., system 100) for verification of the wavefront data from a Shack-Hartmann wavefront sensor.

User interface 400 shows an image 402 collected by the wavefront sensor for an eye, where the image 402 typically includes a plurality of spots 412 (formed by the array of lenses 132). The image 402 includes a plurality of segment indicators 410, shown in FIG. 4 as squares. A respective segment indicator 410 has a corresponding quality indicator (e.g., marking and/or color) that indicates whether or not a respective segment of the image 402 within the segment indicator 410 contains sufficient information that may be used for wavefront analysis. For example, when the respective segment includes a single spot 412, the respective segment indicator 410 may be displayed with a certain color (e.g., green), which indicates successful collection of a corresponding portion of wavefront data. Other indicators 414, such as a cross or an “X”, may be used to indicate that the respective segment may not be useable for wavefront analysis (e.g., the respective segment does not contain any spot or contains two or more spots).

User interface 400 may also include an affordance 420 (e.g., on-screen command, a button, etc.), which, when selected (or activated) by the operator, causes the measurement device 102 to repeat (e.g., restart, re-do) the wavefront sensing operation. In some embodiments, the vision characterization system 100 can perform an analysis of the captured wavefront data (e.g., wavefront sensor image, wavefront information) and can generate an indication 422 of the quality (e.g. Good, Fair, Poor) of the wavefront data or wavefront results (e.g., based on a number of acceptable segments out of all the segments). User interface 400 may also provide an affordance 424 (e.g., on-screen command, a button, etc.), which, when selected by the operator, causes user interface 400 to proceed to a next step or next screen.

As used herein, an affordance is deemed to be selected (or activated) when the system receives a user input at a location that corresponds to the affordance (e.g., a touch input at a touch-sensitive surface, such as a touch-sensitive screen or a touch-sensitive surface of a touch pad device, at a location that corresponds to the location of the affordance in the user interface or a user input, such as a mouse click or a keyboard input, while a location indicator, such as a cursor, is displayed in the user interface at a location that corresponds to the affordance. In some embodiments, in response to receiving a user input, the system processes instructions associated with the affordance (e.g., in response to receiving a touch input 405 at a location that corresponds to affordance 424, the system processes instructions for proceeding to a next step or next screen, such as instructions in user input module 236 and/or vision characterization application 218).

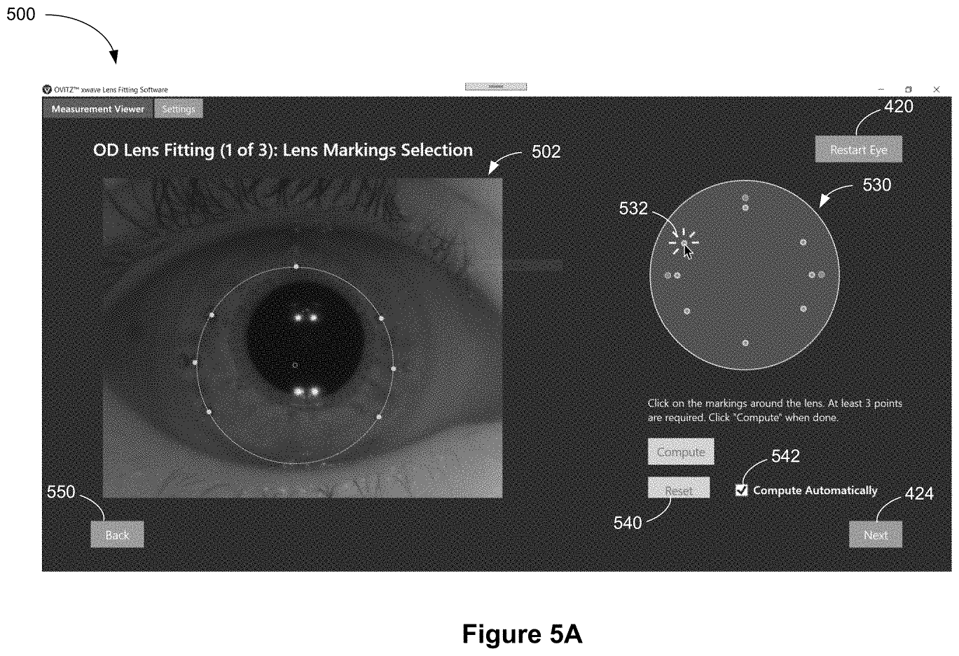

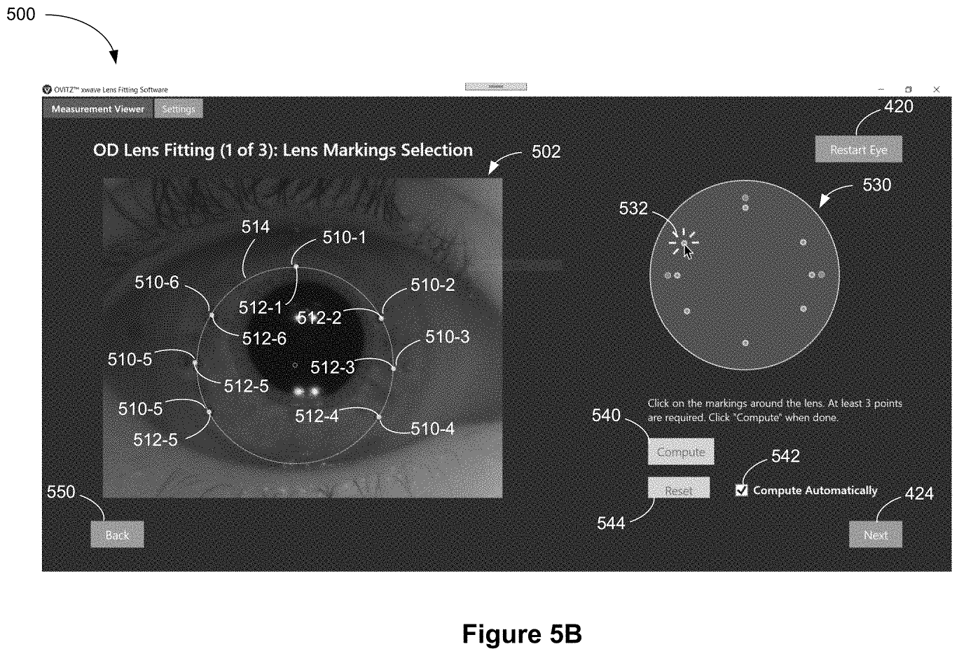

FIGS. 5A and 5B illustrate user interface 500 for identification of markers on a lens in accordance with some embodiments. User interface 500 shows an image 502 of the patient's eye that is captured by the measurement device 102 while the patient is wearing a predicate lens with a plurality of reference markings (e.g., periphery reference markings 510-1 through 510-6, and in some cases, angular reference markings). The image 502 includes at least a portion of the patient's eye including the patient's pupil. In some cases, as shown in FIG. 5B, one or more indicators 512 (e.g., dots) indicating positions of periphery reference markings 510 may be overlaid on top of the image 502. In some cases, the indicators 512 correspond to positions of periphery reference markings 510 that have been identified (e.g., automatically identified or identified independently of user input or manual entry) by the system 100. In FIG. 5B, indicators 512-1 through 512-6 show the positions of corresponding periphery reference markings 510-1 through 510-6 that have been identified by the system 100. In some cases, the indicators 512 correspond to positions of periphery reference markings 510 that have been identified by the user of the system 100.

In some embodiments, user interface 500 also includes a schematic representation 530 of the plurality of markings of the predicate lens. In some embodiments, the system 100 may receive user selection of a periphery reference marking on the schematic representation 530 followed by user selection of a position on the image 502 to indicate a position of the selected periphery reference marking or a correction to an independently identified position of the selected periphery reference marking. For example, the user may select indicator 532 corresponding to periphery reference marking 510-6. If the system 100 did not independently identify a position of periphery reference marking 510-6 yet, the user may manually select a position on the image 502 that corresponds to a position of periphery reference marking 510-6. The system receives the user selection of the position on the image 502 and associates it with the position of periphery reference marking 510-6. In some embodiments, the system 100 may receive user selection of a position on the image 502 followed by user selection of a periphery reference marking on the schematic representation 530 to indicate a position of the selected periphery reference marking or a correction to an independently identified position of the selected periphery reference marking.

In some embodiments, once positions of a certain number (e.g., a number corresponding to a threshold number, such as three) of periphery reference markings have been identified, user interface 500 is updated to include an affordance 540 or an affordance 542 (e.g., on-screen command, a button, etc.), which, when selected by the operator, causes the system 100 to compute or determine a position of the predicate lens. In some embodiments, a circle 514 circumscribing (or substantially circumscribing) the periphery reference markings, whose positions have been identified, is overlaid on top of the image 502. In some embodiments, the system 100 identifies the minimum number of points (e.g., once 3 points are verified as corresponding to a respective periphery reference marking), the system 100 determines and displays (e.g., overlays) a circle 514 circumscribing the position-identified periphery reference markings (or indicators 512) on image 502. In some embodiments, a center of the circle 514 corresponds to a center of the predicate lens (and is deemed to be a representative position of the lens in some cases). As shown in FIG. 5B, in some cases, the center of the predicate lens does not correspond to the center of the pupil of the eye.

In some embodiments, user interface 500 includes an affordance 544 (e.g., on-screen command, a button, etc.), which, when selected by the user, causes identified position(s) of the one or more periphery reference markings 510 to be reset. In some embodiments, affordance 544 (e.g., on-screen command, a button, etc.), when selected by the user, causes the measurement device 102 to re-attempt to locate position(s) of the one or more periphery reference markings 510 in the image 502.

In some embodiments, user interface 500 may include an affordance 550 (e.g., on-screen command, a button, etc.), which, when selected by the user, causes the system 100 to display a user interface for a previous step or previous page (e.g., user interface 400).

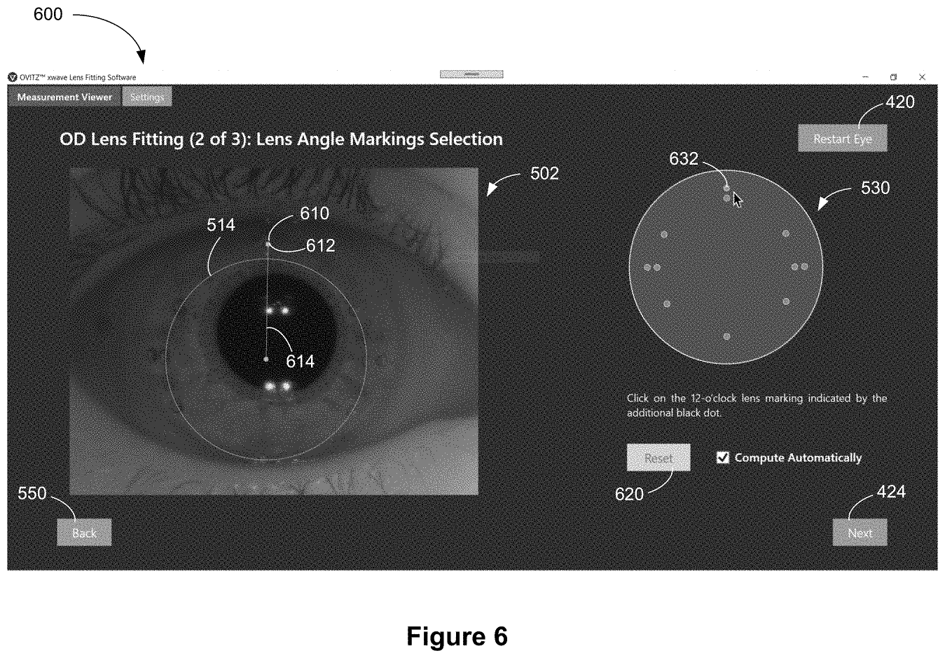

FIG. 6 illustrates user interface 600 for identification of lens markings for angular orientation in accordance with some embodiments. User interface 600 includes image 502, details of which are described above with respect to FIG. 5B, without indicators 512. In user interface 600, one or more indicators 612 (e.g., dots) indicating position(s) of one or more angular reference markings 610 are overlaid on top of the image 502. In some cases, an indicator 612 corresponds to a position of an angular reference marking that has been identified (e.g., automatically identified or identified independently of user input or manual entry, identified via automated detection) by the system 100. For example, FIG. 6 shows indicator 612 identifying the position of angular reference marking 610. In some cases, the indicators 612 correspond to positions of angular reference markings 610 that have been identified by the user of the system 100.

In some embodiments, user interface 600 may also include an affordance 620 (e.g., on-screen command, a button, etc.), which, when selected by the user, causes identified position(s) of the one or more angular reference markings to be reset. In some embodiments, the affordance 620 (e.g., on-screen command, a button, etc.), when selected by the user, causes the measurement device 102 to re-attempt to locate position(s) of the one or more angular reference markings in the image 502.

In some embodiments, the schematic representation 530 also shows the one or more angular reference markings of the predicate lens (in addition to the one or more periphery reference markings). In some embodiments, the system 100 may receive user selection of an angular reference marking on the schematic representation 530 followed by user selection of a position on the image 502 to indicate a position of the selected angular reference marking or a correction to an independently identified position of the selected angular reference marking. For example, the user may select an indicator 632 corresponding to angular reference marking 610. In the case where the system 100 does not independently identify a position of angular reference marking 610 or identified an incorrect position, the user may select a position on the image 502 that corresponds to a correct position of angular reference marking 610. In some embodiments, the system 100 may receive user selection of a position on the image 502 followed by user selection of an angular reference marking on the schematic representation 530 to indicate a position of the selected angular reference marking 610 or a correction to an independently identified position of the selected angular reference marking 610.

In some embodiments, such as when one or more angular reference markings are not visible in the image 502, the operator may select affordance 420, as described above with respect to FIG. 4, to re-image the patient's eye.

In some embodiments, the vision characterization system 100 also identifies (e.g., automatically identifies, identifies independently of user input) and displays a line 614 that passes through at least one angular reference marking of the one or more angular reference markings, such as zero-degree or twelve-o'clock angular reference marking (e.g., line 614 passes through both a center of the lens, represented by a green dot, and angular reference marking 610).

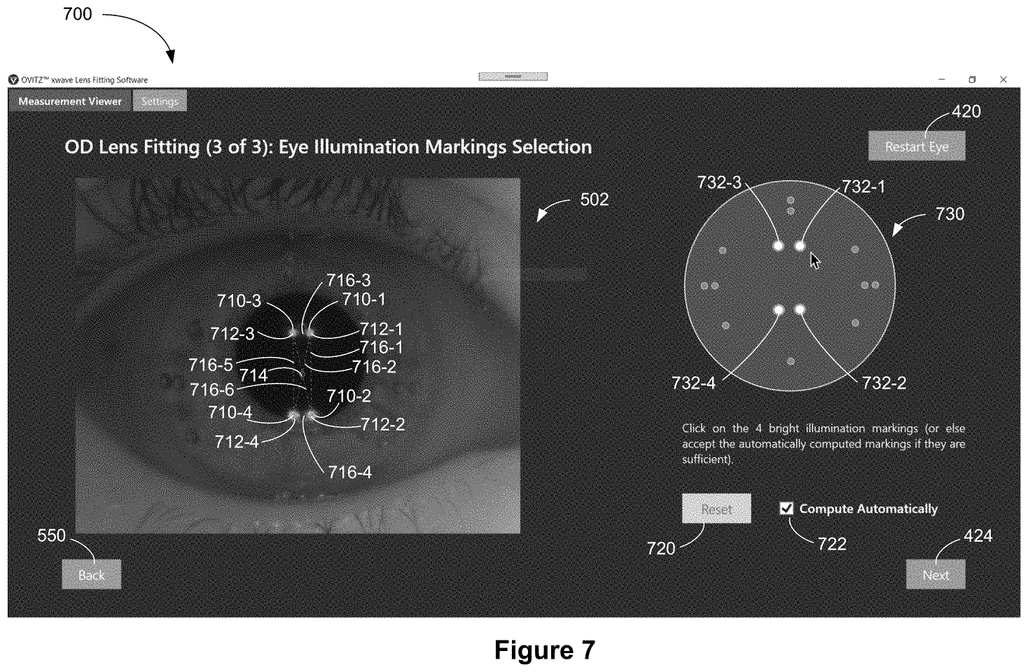

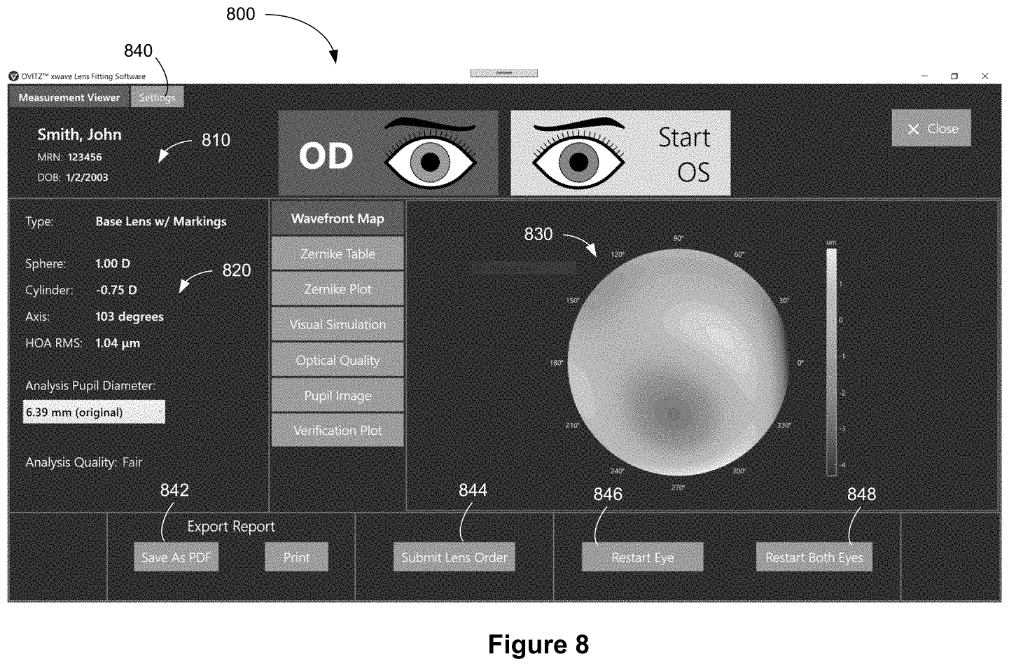

FIG. 7 illustrates user interface 700 for identifying a position reference point, such as identifying the corneal vertex or a visual axis in accordance with some embodiments. User interface 700 includes image 502, details of which are described above with respect to FIGS. 5A, 5B, and 6, without indicators 512 and 612. Alternatively, user interface 700 may show, instead of image 502, another image that is different from image 502 (e.g., an enlarged view of image 502 or a different image). As shown in FIG. 7, image 502 includes reflection of illumination on the patient's eye (e.g., reflection of light from illumination sources in the measurement device 102 shown in FIGS. 1A-1G). In some cases, the vertex of the cornea lies precisely at a central point relative to the reflected light pattern. The position of the vertex of the cornea may be used to approximate a location of a visual axis.

One or more indicators 712 (e.g., indicators 712-1 through 714-4 each having a shape of, for example, a dot) indicating positions of illumination markings 710 (e.g., reflections of the illumination on the patient's eye) are overlaid on the displayed image 502. In some cases, the indicators 712 correspond to positions of illumination markings 710 that have been identified (e.g., automatically identified or identified independently of user input or manual entry, identified via automated detection) by the system 100. For example, FIG. 7 shows that indicators 712-1 through 712-2 indicate the positions of the illumination markings 710-1 through 710-4. In some cases, the indicators 712 correspond to positions of illumination markings 710 that have been identified manually.

As shown in FIG. 7, in some embodiments, user interface 700 also includes lines 716-1 through 716-6, each of which connects a respective pair of the two or more illumination markings (e.g., line 716-1 connects illumination markings 710-1 and 710-2, line 716-2 connect illumination markings 710-2 and 710-3, and so on).

In some embodiments, user interface 700 includes indicator 714, which represents a center of illumination markings 710-1 through 710-4. In some cases, the center of illumination markings 710-1 through 710-4 is deemed to be a position reference point of the eye (e.g., a visual axis or a corneal vertex of the eye).

In some cases, as shown in FIG. 7, the corneal vertex has a position that is different from (e.g., noticeably displaced from) the center of the pupil of the imaged eye. Thus, in some embodiments, user interface 700 is used to identify a position of a pupil center. For example, user interface 700 includes indication of a boundary of a pupil region and a center of the pupil region that have been automatically identified. Alternatively, user interface 700 is used to receive user indication of positions along the boundary of the pupil region, from which a center of the pupil region is determined. In some cases, the pupil center is used as a position reference point of the eye. For brevity, such details are omitted herein.

In some embodiments, user interface 700 also includes a schematic representation 730 of the illumination markings (e.g., indicators 732-1 through 732-4 corresponding to illumination markings). In some embodiments, the system 100 may receive user selection of an indicator 732 on the schematic representation 730 followed by user selection of a position on the displayed image to indicate a position of the selected illumination marking 710 or a correction to an independently identified position of the selected illumination marking 710. For example, the user may select an indicator 732-1 corresponding to a first illumination marking 710-1. In the case where the vision characterization system 100 did not identify independently a position of the first illumination marking 710-1, or identified an incorrect position, the user may select a position on the image 502 that corresponds to a position of the first illumination marking 710-1. In some embodiments, the system 100 may receive user selection of a position on the displayed image to indicate a position of the selected illumination marking 710 followed by user selection of an indicator 732 on the schematic representation 730.

User interface 700 may also include an affordance 722 (e.g., on-screen command, a button, etc.), which, when selected by the operator, causes the system 100 to automatically determine (e.g., location or compute) a position of the vertex of the cornea based on the determined (e.g., verified or confirmed) positions of the illumination markings shown in the displayed image (e.g., image 502).

In some embodiments, user interface 700 also includes an affordance 720 (e.g., on-screen command, a button, etc.), which, when selected by the operator, causes the measurement device 102 to re-attempt to locate positions of the illumination markings in the displayed image (e.g., image 502). In some embodiments, the affordance 720, when selected by the operator, causes identified positions of the illumination markings to be reset.

In some embodiments, such as when one or more illumination markings are not visible in the displayed image (e.g., image 502), the operator may select affordance 420, as described above with respect to FIG. 4, to re-image the patient's eye.

In some embodiments, user interface 700 includes an affordance, which, when selected, causes the system to use calculated or automatically identified values, rather than prompting for user selection.

In some embodiments, one or more of user interfaces 500, 600, 700 include instructions for how to navigate, use, or interact with such user interfaces.

FIG. 8 illustrates summary screen user interface 800 for user review, reporting, and file generation in accordance with some embodiments. User interface 800 includes a summary screen for a user, operator, practitioner, or optometrist to review the results, report the results, or generate and save a file with the results. User interface 800 displays information regarding the patient 810 and/or results 820 from the vision characterization process. User interface 800 also displays a graphical representation 830 of the results of the vision characterization (e.g., waterfront sensing) and/or a lens profile.

User interface 800 also provides a number of operator selections, such as an option 840 to specify, view data, and/or view aberration characteristics for either eye of the patient; an option 842 to generate reports, and an option 844 to export the lens fabrication file to another system or to a memory. For example, the lens fabrication file may be transferred to another computer system, such as a lens fabrication system or a lens order system for placing a lens order. Alternatively, the lens fabrication file may be saved locally.

User interface 800 may also provide an option 846 and/or 848 to restart one or both eyes.

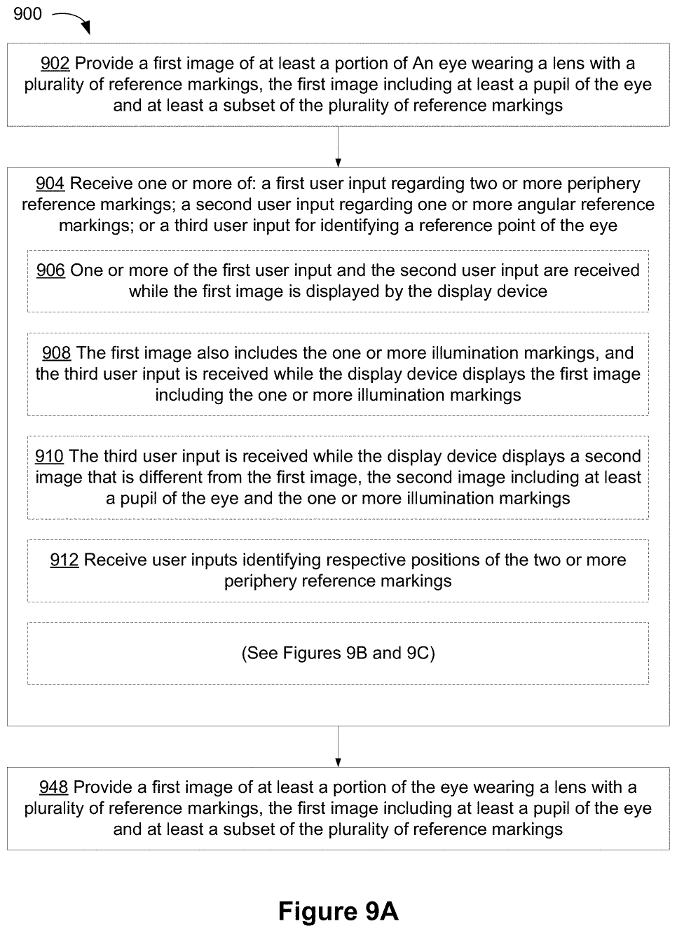

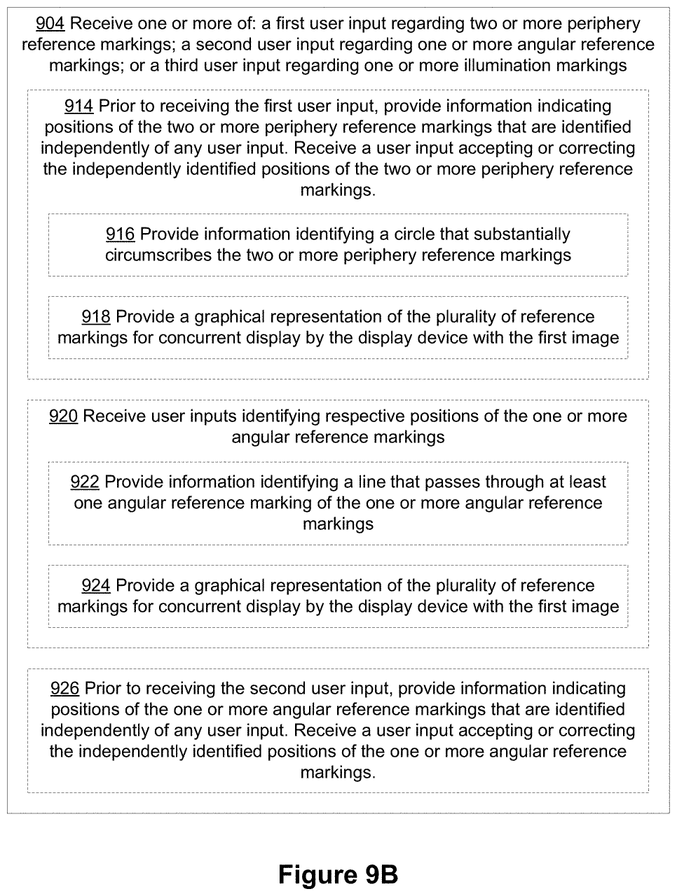

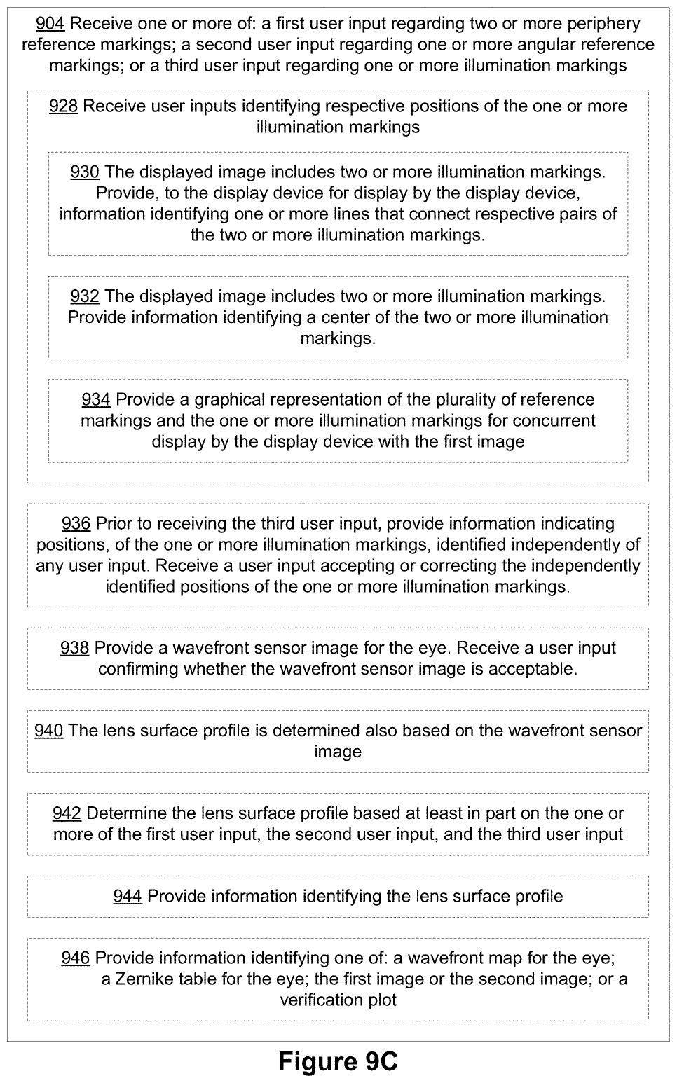

FIGS. 9A-9C illustrate a flow diagram representing a method 900 for characterizing an eye of a user in accordance with some embodiments. In some embodiments, method 900 is performed by the system 104 (or more generally the system 100).

Method 900 includes (902) providing, to the display device (e.g., display device 108) for display by the display device, a first image (e.g., image 502) of at least a portion of the eye wearing a lens (e.g., a predicate lens) with a plurality of reference markings (e.g., periphery reference markings 510, angular reference markings 610, illumination markings 710). The first image includes at least a pupil of the eye and at least a subset of the plurality of reference markings (e.g., in some cases, one or more of the plurality of reference markings on the lens may be blocked in the image).

Method 900 also includes (904) receiving one or more of a first user input regarding two or more periphery reference markings (e.g., a user input identifying, correcting, or confirming positions of two or more periphery reference markings), a second user input regarding one or more angular reference markings (e.g., a user input identifying, correcting, or confirming positions of one or more or more angular reference markings), or a third user input regarding a position reference point (e.g., a user input identifying, correcting, or confirming positions of one or more illumination markings, from which a visual axis of the eye is determined, or two or more positions along a boundary of a pupil, from which a pupil center is determined).

In some embodiments, lens is a contact lens (e.g., a scleral lens).

In some embodiments, the lens is an optically transparent lens and the plurality of reference markings is visually distinguishable from the optically transparent lens. For example, the plurality of reference markings may have optical properties (e.g., opaqueness and/or color) that are visually distinguishable from those of the optically transparent lens. A respective reference marking may have a shape of a dot, a rectangle, an ellipse, a circle, or any other object.

In some embodiments, the image is collected from the eye wearing the lens with the plurality of reference markings while the eye is illuminated with an illumination pattern that provides the one or more illumination markings (e.g., reflection of the illumination pattern) on the eye. For example, the image 502 shown in FIG. 5B is taken while the eye wearing a lens with a plurality of reference markings (e.g., periphery reference markings 510 and angular reference marking 610) is illuminated with an illumination pattern (e.g., four-spot illumination) so that illumination markings 710-1 through 710-4 are captured in the image 502.

In some embodiments, the second user input is received subsequent to receiving the first user input (e.g., after a user input is received with respect to the user interface 500, the user interface 500 is replaced with the user interface 600 for receiving a user input regarding one or more angular reference markings). In some embodiments, the first user input is received subsequent to receiving the second user input.

In some embodiments, the third user input is received subsequent to receiving the second user input (e.g., after a user input is received with respect to the user interface 600, the user interface 600 is replaced with the user interface 700 for receiving a user input regarding one or more illumination markings). In some embodiments, the second user input is received subsequent to receiving the third user input.

In some embodiments, the first user input is received subsequent to receiving the third user input. In some embodiments, the third user input is received subsequent to receiving the first user input.

In some embodiments, method 900 includes receiving two or more of the first user input, the second user input, or the third user input (e.g., the first user input and the second user input, the first user input and the third user input, or the second user input and the third user input).

In some embodiments, method 900 includes receiving all three of the first user input, the second user input, and the third user input (e.g., the user interface 500, 600, and 700 are displayed in sequence for receiving the first user input, the second user input, and the third user input).

In some embodiments, one or more of the first user input and the second user input are received (906) while the first image is displayed by the display device (e.g., as shown in FIGS. 5A, 5B, and 6, the first user input regarding two or more periphery reference markings and the second user input regarding one or more angular reference markings are received while the image 502 is displayed).

In some embodiments, the first image also includes (908) the one or more illumination markings, and the third user input is received while the display device displays the first image including the one or more illumination markings (e.g., as shown in FIG. 7, the third user input regarding one or more illumination markings is received while the image 502 including illumination markings 710-1 through 710-4 is displayed).

In some embodiments, the third user input is received (910) while the display device displays a second image that is different from the first image. The second image includes at least a pupil of the eye and the one or more illumination markings. For example, the second image may be a subset, less than all, of the first image, where the second image also includes at least a pupil of the eye and the one or more illumination markings. In some cases, the subset of the first image is enlarged in the second image to facilitate accurate identification of the positions of the illumination markings.

In some embodiments, receiving the first user input includes (912) receiving user inputs identifying respective positions of the two or more periphery reference markings (e.g., user inputs identifying positions of two or more of the periphery reference markings 510-1 through 510-6).

In some embodiments, method 900 includes (914, FIG. 9B), prior to receiving the first user input, providing, to the display device for display by the display device, information indicating positions, of the two or more periphery reference markings, that are identified independently of any user input (e.g., in FIG. 5B, indicators 512-2, 512-3, 514-5, and 512-5 may indicate positions of periphery reference markings that are automatically identified by the system 100). In some embodiments, receiving the first user input includes receiving a user input that accepts or corrects the independently identified positions of the two or more periphery reference markings (e.g., a user input selecting or activating the affordance 424 in FIG. 5B).

In some embodiments, the positions, of the two or more periphery reference markings, that are identified independently of any user input are overlaid on the displayed image (e.g., in FIG. 5B, indicators 512-2, 512-3, 514-5, and 512-5, whose positions have been identified by the system 100 without, or before receiving, a user input, are overlaid on the image 502 over corresponding periphery reference markings 510-2, 510-3, 510-4, and 510-5).

In some embodiments, method 900 also includes (916) providing, to the display device for display by the display device, information identifying a circle that substantially circumscribes the two or more periphery reference markings (e.g., circle 514). In some embodiments, the circle circumscribes the two or more periphery reference markings. In some embodiments, the circle is a fit (e.g., a regression fit) to the two or more periphery reference markings. In some embodiments, the circle may not circumscribe any of the two or more periphery reference markings.

In some embodiments, method 900 also includes (918) providing, to the display device, a graphical representation (e.g., schematic representation 530 shown in FIG. 5B) of the plurality of reference markings for concurrent display by the display device with the first image. This facilitates user identification of positions of markings by providing a visual reference or by allowing the user to select a particular marking before or after providing position information.

In some embodiments, the two or more periphery reference markings are visually highlighted in the graphical representation (e.g., the graphical representation 530 in FIG. 5B includes periphery reference markings and angular reference markings and the periphery reference markings are highlighted in yellow).

In some embodiments, receiving the second user input includes (920) receiving user inputs identifying respective positions of the one or more angular reference markings (e.g., user inputs identifying positions of one or more of the angular reference markings, such as the angular reference marking 610 shown in FIG. 6).

In some embodiments, method 900 also includes (922) providing, to the display device for display by the display device, information identifying a line that passes through at least one angular reference marking of the one or more angular reference markings (e.g., line 614). In some embodiments, the line also passes through a center of the plurality of periphery reference markings (or a center of the circle 514).

In some embodiments, method 900 also includes (924) providing, to the di splay device, a graphical representation (e.g., schematic representation 530 shown in FIG. 6) of the plurality of reference markings for concurrent display by the display device with the first image.

In some embodiments, the one or more angular reference markings are visually highlighted in the graphical representation (e.g., the graphical representation 530 in FIG. 6 includes periphery reference markings and angular reference markings and an angular reference marking is highlighted in green).

In some embodiments, method 900 further includes, (926) prior to receiving the second user input, providing, to the display device for display by the display device, information indicating positions, of the one or more angular reference markings, that are identified independently of any user input (e.g., in FIG. 6, indicator 612 may indicate a position of an angular reference marking that has been automatically identified by the system 100). In some embodiments, receiving the second user input includes receiving a user input that accepts or corrects the independently identified positions of the one or more angular reference markings (e.g., a user input selecting or activating the affordance 424 in FIG. 6).

In some embodiments, the positions, of the one or more angular reference markings, that are identified independently of any user input are overlaid on the first image (e.g., in FIG. 6, indicator 612, whose position has been identified by the system 100 without, or before receiving, a user input, is overlaid on the image 502 over corresponding angular reference marking 610).

In some embodiments, receiving the third user input includes (928, FIG. 9C) receiving user inputs identifying respective positions of the one or more illumination markings (e.g., user inputs identifying positions of the one or more illumination markings 710-1 through 710-4 shown in FIG. 7).

In some embodiments, the positions, of the one or more illumination markings, identified independently of any user input are overlaid on the image including the at least a pupil of the eye and the at least a subset of the plurality of reference markings (e.g., in FIG. 7, indicators 712-1 through 712-4, whose positions have been identified by the system 100 without, or before receiving, a user input, is overlaid on the image 502 over corresponding illumination markings 710-1 through 710-4).

In some embodiments, the displayed image (e.g., the first image or the second image) includes (930) two or more illumination markings, and method 900 also includes providing, to the display device for display by the display device, information identifying one or more lines that connect respective pairs of the two or more illumination markings (e.g., lines 716-1 through 716-6).

In some embodiments, the displayed image (e.g., the first image or the second image) includes (932) two or more illumination markings, and method also includes providing, to the display device for display by the display device, information identifying a center of the two or more illumination markings (e.g., indicator 714).

In some embodiments, method 900 also includes (934) providing, to the display device, a graphical representation of the plurality of reference markings and the one or more illumination markings for concurrent display by the display device with the first image (or the second image). For example, in FIG. 7, the graphical representation 730 including the plurality of reference markings (e.g., the periphery reference markings and the angular reference markings) and the illumination markings is displayed concurrently with the image 502.

In some embodiments, the one or more illumination markings are visually highlighted in the graphical representation (e.g., the graphical representation 730 includes the plurality of periphery reference markings and angular reference markings, and illumination markings that are highlighted in blue).

In some embodiments, method 900 further includes, (936) prior to receiving the third user input, providing, to the display device for display by the display device, information indicating positions, of the one or more illumination markings, identified independently of any user input (e.g., in FIG. 7, indicators 712-1 through 714-4 may indicate positions of illumination markings that have been automatically identified by the system 100). Receiving the third user input includes receiving a user input accepting or correcting the independently identified positions of the one or more illumination markings (e.g., a user input selecting or activating the affordance 424 in FIG. 7).

In some embodiments, method 900 also includes (938) providing, to the display device for display by the display device, a wavefront sensor image for the eye and receiving a user input confirming whether the wavefront sensor image is acceptable For example, in FIG. 4, the image 402 containing a wavefront sensor image is provided to the display device for display and the system receives a user input, such as input 405, at a location that corresponds to affordance 424, which indicates that the wavefront sensor image is acceptable. Alternatively, the system may receive a user input at a location that corresponds to affordance 420, which indicates that the wavefront sensor image is not acceptable.

In some embodiments, a respective region of a plurality of regions of the wavefront sensor image includes an indication of whether the respective region satisfies predefined acceptance criteria. In some embodiments, the system automatically initiates retaking a wavefront sensor image in accordance with a determination that the number of regions of the wavefront sensor image that do not satisfy the predefined acceptance criteria is greater than a predefined threshold.

In some embodiments, the indication is any of: a graphical indication, a marker that indicates, color coded indicator, and a coded symbol. For example, in FIG. 4, regions of the wavefront sensor image that do not satisfy the predefined acceptance criteria are shown with a red “X” mark and regions of the wavefront sensor image that satisfy the predefined acceptance criteria are shown with a green dot.

In some embodiments, a respective region of the plurality of regions of the wavefront sensor image is distinct from and does not overlap with another region of the plurality of regions of the wavefront sensor image.

In some embodiments, the lens surface profile (940) is determined also based on the wavefront sensor image. The method of determining a lens surface profile is described in U.S. patent application Ser. No. 16/558,298, which is incorporated by reference herein in its entirety.

In some embodiments, obtaining the lens surface profile includes (942) determining the lens surface profile based at least in part on the one or more of the first user input, the second user input, and the third user input. For example, the position of the lens relative to the center of the pupil or the visual axis of the eye is determined based on the positions of the reference markings and illumination markings, and the relative position of the lens is used to offset the lens surface profile (or a component of the surface profile used for compensating for higher-order aberrations).

In some embodiments, method 900 also includes (944) providing, to the display device for display by the display device, information identifying the lens surface profile. In some cases, the lens surface profile is displayed as a contour map. In some other cases, a cross-section of the lens surface profile is displayed.

In some embodiments, method 900 also includes (946) providing, to the display device for display by the display device, information identifying one of: a wavefront map for the eye, a Zernike table for the eye, the first image or the second image, or a verification plot. In some embodiments, the system provides to the display device information identifying a user interface with a plurality of affordances, a respective affordance of the plurality of affordances, which, when selected (or activated), causes the system to provide to the display device information identifying one of: a wavefront map for the eye, a Zernike table for the eye, the first image or the second image, or a verification plot so that the user interface with the plurality of affordances is replaced with the selected information. Alternatively, the selected information is displayed within the same user interface including the plurality of affordances.

Method 900 further includes (948, FIG. 9A) obtaining a lens surface profile determined based at least in part on the one or more of the first user input, the second user input, and the third user input.

The foregoing description, for purpose of explanation, has been described with reference to specific embodiments. However, the illustrative discussions above are not intended to be exhaustive or to limit the scope of claims to the precise forms disclosed. Many modifications and variations are possible in view of the above teachings. For example, the methods described above may be used for designing and making lenses for spectacles (e.g., eyeglasses). The embodiments were chosen and described in order to best explain the principles of the various described embodiments and their practical applications, to thereby enable others skilled in the art to best utilize the invention and the various described embodiments with various modifications as are suited to the particular use contemplated.

What is claimed is:

1. A method for characterizing an eye of a user, the method comprising:
at an electronic device with one or more processors and memory, the electronic device being in communication with a display device:
providing, to the display device for display by the display device, a first image of at least a portion of the eye wearing a lens with a plurality of reference markings, the first image including at least a pupil of the eye and at least a subset of the plurality of reference markings;
receiving one or more of:
a first user input regarding two or more periphery reference markings;
a second user input regarding one or more angular reference markings; or
a third user input for identifying a position reference point of the eye; and
obtaining a lens surface profile determined based at least in part on the one or more of the first user input, the second user input, and the third user input.

2. The method of claim 1, wherein one or more of the first user input and the second user input are received while the first image is displayed by the display device.

3. The method of claim 1, wherein the first image also includes one or more illumination markings, and the third user input is received while the display device displays the first image including the one or more illumination markings.

4. The method of claim 1, wherein the third user input is received while the display device displays a second image that is different from the first image, the second image including at least a pupil of the eye and one or more illumination markings.

5. The method of claim 1, wherein:
receiving the first user input includes receiving user inputs identifying respective positions of the two or more periphery reference markings.

6. The method of claim 5, further comprising:
providing, to the display device for display by the display device, information identifying a circle that substantially circumscribes the two or more periphery reference markings.

7. The method of claim 5, further comprising:
providing, to the display device, a graphical representation of the plurality of reference markings for concurrent display by the display device with the first image.

8. The method of claim 1, further comprising:
prior to receiving the first user input:
providing, to the display device for display by the display device, information indicating positions of the two or more periphery reference markings that are identified independently of any user input, wherein receiving the first user input includes receiving a user input accepting or correcting the independently identified positions of the two or more periphery reference markings.

9. The method of claim 1, wherein:
receiving the second user input includes receiving user inputs identifying respective positions of the one or more angular reference markings.

10. The method of claim 9, further comprising:
providing, to the display device for display by the display device, information identifying a line that passes through at least one angular reference marking of the one or more angular reference markings.

11. The method of claim 9, further comprising:
providing, to the display device, a graphical representation of the plurality of reference markings for concurrent display by the display device with the first image.

12. The method of claim 1, further comprising:
prior to receiving the second user input:
providing, to the display device for display by the display device, information indicating positions of the one or more angular reference markings that are identified independently of any user input, wherein receiving the second user input includes receiving a user input accepting or correcting the independently identified positions of the one or more angular reference markings.

13. The method of claim 1, wherein:
receiving the third user input includes receiving user inputs identifying respective positions of one or more illumination markings.

14. The method of claim 13, wherein:
the displayed image includes two or more illumination markings; and
the method further includes providing, to the display device for display by the display device, at least one of:
information identifying one or more lines that connect respective pairs of the two or more illumination markings; or
information identifying a center of the two or more illumination markings.

15. The method of claim 13, further comprising:
providing, to the display device, a graphical representation of the plurality of reference markings and the one or more illumination markings for concurrent display by the display device with the first image.

16. The method of claim 1, further comprising:
prior to receiving the third user input:
providing, to the display device for display by the display device, information indicating positions, of one or more illumination markings, identified independently of any user input, wherein receiving the third user input includes receiving a user input accepting or correcting the independently identified positions of the one or more illumination markings.

17. The method of claim 1, further comprising:
providing, to the display device for display by the display device, a wavefront sensor image for the eye; and
receiving a user input confirming whether the wavefront sensor image is acceptable.

18. The method of claim 1, wherein:
the lens surface profile is determined also based on the wavefront sensor image.

19. The method of claim 1, wherein:
obtaining the lens surface profile includes determining the lens surface profile based at least in part on the one or more of the first user input, the second user input, and the third user input.

20. The method of claim 1, further comprising:
providing, to the display device for display by the display device, information identifying one of:
the lens surface profile;
a wavefront map for the eye;
a Zernike table for the eye;
the first image or the second image; or
a verification plot.
